(19) United States
(12) Patent Application Publication
(10) Pub. No.: US 2018/0009903 A1
(43) Pub. Date: Jan. 11, 2018

(54) MODULATING ADIPOSE TISSUE AND ADIPOGENESIS

(71) Applicant: Katholieke Universiteit Leuven Ku Leuven Research & Development, Leuven (BE)

(72) Inventors: Henri Roger Lijnen, Herent (BE); Dries Bauters, Sint-Truiden (BE)

(21) Appl. No.: 15/520,285

(22) PCT Filed: Oct. 22, 2015
(86) PCT No.: PCT/BE2015/000061

§ 371 Date: Apr. 19, 2017

Related U.S. Application Data
(60) Provisional application No. 62/090,283, filed on Dec. 10, 2014.
(60) Provisional application No. 62/090,280, filed on Dec. 10, 2014.
(60) Provisional application No. 62/072,769, filed on Oct. 30, 2014.
(60) Provisional application No. 62/067,383, filed on Oct. 22, 2014.

(51) Int. Cl. C07K 16/40

(52) U.S. Cl. CPC C07K 16/40; C12Y 304/24; C07K 2317/76

(57) ABSTRACT

The invention relates to the field of obesity and related metabolic diseases. More specifically, the invention relates to methods of reducing aggrecanase activity or antigen in mammals in order to enhance brown adipose tissue (BAT) development, to promote conversion of white adipose tissue (WAT) into BAT in vivo, and to limit triglyceride accumulation and steatosis in the liver. The invention also relates to a strategy of neutralization or depletion of ADAMTS5 as a strategy to inhibit adipogenesis and more specifically, the invention relates to a method of reducing aggracanase-2 (ADAMTS5, A Disintegrin and Metalloproteinase with Thrombospondin motif 1; member 5) antigen and/or activity in mammals in order to impair differentiation of precursor cells into mature adipocytes (i.e., adipogenesis).

### BACKGROUND AND SUMMARY

### BACKGROUND OF THE DISCLOSURE

### A. Field of the disclosure

The disclosure relates to the field of obesity and related metabolic diseases, including type 2 diabetes, insulin resistance, atherosclerosis and lipid disorders. More specifically, the disclosure relates to aggrecanase 2 (ADAMTS5 or ADAM-TS5), a disintegrin and metalloproteinase with thrombospondin motif 1; member 5, and more specifically methods of neutralising, depleting or reducing ADAMTS5 activity or ADAMTS5 antigen in mammals for one of the following: to enhance brown adipose tissue (BAT) development, to limit white adipose tissue (WAT) formation, to promote conversion of white adipose tissue (WAT) into BAT in vivo, to limit triglyceride accumulation and steatosis in the liver (hepatosteatosis), to treat nonalcoholic fatty liver disease (ICD10 (2015) K76.0)), lipid storage disorder (ICD10 (2015) E75.6), to inhibit adipogenesis and more specifically to impair differentiation of precursor cells into mature adipocytes (i.e., adipogenesis) and to treat disorders of lipoprotein metabolism and other lipidaemia (ICD10 (2015) E78).

### B. Brief Description of the Related Art and Our Disclosure

In mammals, there are two types of adipose tissue: white adipose tissue (WAT) as the site of energy storage and brown adipose tissue (BAT) that dissipates energy as heat. Expansion of WAT is supported either by new adipocyte differentiation (hyperplasia) or by existing adipocyte expansion (hypertrophy).

Whereas adipocytes in WAT contain one large globule of triglycerides, BAT contains numerous smaller droplets of triglycerides, more mitochondria and a specific uncoupling protein 1 (UCP1) that regulates conversion of energy into heat by uncoupling ATP production from mitochondrial respiration. In man, BAT mass proportionally decreases when body mass index increases, and obese individuals have lower levels of UCP1. Studies in both animals and humans suggest that BAT and mitochondrial uncoupling are potential interventional targets to prevent and treat obesity and related metabolic disorders (reviewed in Ginter E, Simko V Brown fat tissue—a potential target to combat obesity. Bratisl Lek Listy. 2012; 113(1):52-6, Saito M. Human brown adipose tissue: regulation and anti-obesity potential. Endocr J 2014; 61(5):409-16, Loyd C, Obici S. Brown fat fuel use and regulation of energy homeostasis. Curr Opin Clin Nutr Metab Care. 2014; 17(4):368-72 and Lidell M E, Betz M J, Enerbäck S. Brown adipose tissue and its therapeutic potential. J Intern Med. 2014; 276(4):364-77). As an alternative to increasing BAT mass, conversion of WAT into BAT results in altering the adipocyte phenotype from fatty acid storage to its utilization, and to improve metabolic complications of obesity (Langin D. Recruitment of brown fat and conversion of white into brown adipocytes: strategies to fight the metabolic complications of obesity? Biochim Biophys Acta. 2010; 1801(3):372-6.). UCP1 positive cells that emerge in WAT under specific stimuli, are termed beige or brite cells. It was shown that inhibition of beige adipocyte function in subcutaneous fat is associated with obesity, insulin resistance and hepatic steatosis (Cohen P, Levy J D, Zhang Y, Frontini A, Kolodin D P, Svensson K J, et al. Ablation of PRDM16 and beige adipose causes metabolic dysfunction and a subcutaneous to visceral fat switch. Cell 2014; 156, 304-16). Induction of BAT can be achieved by stimuli such as cold exposure or via activation of several different pathways (Ginter E, Simko V. Brown fat tissue—a potential target to combat obesity. Bratisl Lek Listy. 2012; 113(1):52-6, Saito M Human brown adipose tissue: regulation and anti-obesity potential. Endocr 1 2014; 61(5):409-16), but well-defined and effective metabolic and pharmacologic interventions are lacking.

Our disclosure proposes neutralization of activity or antigen of aggrecanase 2 (ADAMTS5 or ADAM-TS5) as a strategy to enhance de novo BAT development, to limit WAT formation and to convert WAT in BAT in vivo for instance to target metabolic disturbances, including type 2 diabetes, insulin resistance, atherosclerosis and lipid disorders. Furthermore, ADAMTS5 neutralization or depletion is a strategy to limit new adipocyte differentiation and to prevent triglyceride accumulation in the liver (steatosis). ADAMTS5 cleaves aggrecan in its interglobular domain. A potential role of aggrecanases in induction of BAT, conversion of WAT into BAT or storage of triglycerides has, however, not been reported.

It is known in the art that the development of obesity is associated with substantial modulation of adipose tissue structure, involving adipogenesis, angiogenesis and extracellular matrix remodelling. Expansion of adipose tissue is supported either by new adipocyte differentiation (hyperplasia) or by expansion of existing adipocytes (hypertrophy).

The generation of mature adipocytes comprises two phases: determination and differentiation. In this process, multipotent stem cells become adipoblasts that can further differentiate info pre-adipocytes, cells already committed to become fat cells. Under appropriate stimulation, in the ultimate phase of differentiation, pre-adipocytes convert to mature, lipid-laden adipocytes. The first phase, determination, involves the commitment of multipotent stem cells to the adipocyte lineage, at which point the cell is referred to as a pre-adipocyte. During the second phase, terminal differentiation, the pre-adipocyte undergoes multiple rounds of mitosis before exiting the cell cycle and differentiating into a mature adipocyte. In the progression from pre-adipocyte to adipocyte the cells takes on the characteristics and metabolic capabilities of a mature adipocyte while under the control of a series of tightly regulated transcriptional and morphological changes. White adipose tissue (WAT) is readily available for study from human patient samples and experimental animal models; however, it is difficult to maintain in vitro and cannot be expanded. Terminal differentiation is more extensively characterized in immortalized cell lines, such as the mouse lines 3T3-F442A and 3T3-L1 (Green H, Kehinde O. Spontaneous heritable changes leading to increased adipose conversion in 3T3 cells. Cell 1976; 7: 105-13), which can undergo one or two rounds of cell division prior to differentiation. Pre-adipocytes maintain the ability to divide and have a turnover rate of up to 4.5 and 5% per day for humans and mice, respectively. Mature adipocytes are widely believed to have lost the ability to divide following the completion of terminal differentiation, and adipocyte turnover has been reported to be up to 10% per year (Spalding KL, Amer E, Westermark P O, Bernard S, Buchholz B A, Bergmann O, Blomqvist L, Hoffstedt J, Naslund E, Britton T, Concha H, Hassan M, Ryden M, Frisen J, Amer P. Dynamics of fat cell turnover in humans. Nature. 2008; 453, 783-7).

Our disclosure proposes neutralization of ADAMTS5 antigen or inhibition of its activity as a strategy to impair differentiation of adipose precursor cells into mature adipocytes (hyperplasia). ADAMTS5 cleaves the extracellular matrix component aggrecan in its interglobular domains. The role of aggrecanases in adipocyte differentiation had not yet been reported.

### SUMMARY OF THE INVENTION

The present invention is predicated on the discovery by the inventors that reduction of ADAMTS5 antigen or ADAMTS5 activity induces conversion of white fat (WAT) into BAT, that it impairs differentiation of adipocyte precursor cells into mature adipocytes (hyperplasia), that it prevents or reduces the de novo adipogenesis or de novo fat development and that it effectively in a mammalian subject inhibits or reduces disorders of lipoprotein metabolism or other lipidaemia (ICD10 (2015) E78), lipid storage disorder (ICD10 (2015) E75.6) and triglyceride accumulation and/or steatosis in the liver leading for instance to nonalcoholic fatty liver disease (ICD10 (2015) K76.0)). Neutralization, reduction or inhibition of aggrecanase 2 activity can be achieved by proteinase inhibitors, neutralizing antibodies (polyclonal, monoclonal, any antibody fragment . . . ), any genetic approach (knockdown, siRNA . . . ) or any other means resulting in reduction of aggrecanase 2ADAMTS5 antigen or activity. It was demonstrated that neutralization or depletion of ADAMTS5 activity and specific neutralization or depletion of ADAMTS5 activity or reduction of antigen concentration, impairs differentiation of adipocyte precursor cells into mature adipocytes (hyperplasia) and that its protects against de novo adipogenesis and de novo fat development. Moreover it was demonstrated that neutralization or depletion of ADAMTS5 activity and specific neutralization or depletion of ADAMTS5 activity promotes BAT development, induces conversion of WAT into BAT or promotes BAT development and induces conversion of WAT into BAT. Moreover it protects against triglyceride accumulation and liver steatosis in a mammalian subject upon high fat feeding. Neutralization of aggrecanase activity (ADAMTS4 and ADAMTS5 activity and /or antigen) also has this effect. It is a valid strategy to prevent/treat obesity and related metabolic diseases, such as insulin resistance. Neutralization of aggrecanase activity can be achieved by proteinase inhibitors; neutralizing antibodies (polyclonal, monoclonal, any antibody fragment . . . ), any genetic approach (knockdown, siRNA . . . ) or any other means resulting in reduction of ADAMTS4/ADAMTS5 antigen or activity or more specifically resulting in reduction of ADAMTS5 antigen or activity.

According to the present invention there is provided method of inducing brown fat (BAT) development in a mammalian subject for instance in a human or of treating to impair differentiation of adipocyte precursor cells into mature adipocytes (hyperplasia) in a mammalian subject for instance in a human or of preventing or reducing the de novo adipogenesis or de novo fat development, comprising administering to a subject in need of such treatment, a therapeutically effective amount of a pharmaceutical composition to inhibit or neutralise the activity of ADAMTS5 or to deplete or reduce the amount of ADAMTS5 antigen in said subject. In a practical embodiment is this method according to the present invention comprises an ADAMTS5 activity neutralising or inhibiting composition that comprises an ADAMTS5 antagonist of the group consisting of peptides and peptidomimetics, antibodies, small molecule inhibitors, double-stranded RNA, aptamers and ribozymes. For instance this object of the present invention is achieved by means of an ADAMTS5 activity neutralising or inhibiting composition comprising a specific polyclonal or monoclonal antibody full-length antibodies or binding fragments or surrogates thereof of the group consisting of Fab′, F(ab′)2, Fab, Fv, vIgG, scFv fragments and surrobodies, rIgG, disulfide-stabilized Fv antibodies (dsFv), diabodies, triabodies, and single domain antibodies, such as a camelised antibody or nanobody. In need of such treatment means for instance that the mammalian suffers of one of obesity and related metabolic diseases, including type 2 diabetes, insulin resistance, atherosclerosis or lipid disorders or triglyceride accumulation and/or steatosis in the liver, nonalcoholic fatty liver disease (ICD10 (2015) K76.0)), lipid storage disorder (ICD10 (2015) E75.6), a disorders of lipoprotein metabolism or other lipidaemia (ICD10 (2015) E78). In need of such treatment means also for instance that the subject is in need of treatment for modulation of thermogenesis, the subject is in need of treatment for obesity and/or one or more pathological conditions resulting thereof, the subject is in need of treatment of improving the metabolic profile, the subject is in need of preventing an obesity related metabolic diseases, such as insulin resistance or the subject is in need of protection against triglyceride accumulation and liver steatosis.

This ADAMTS5 activity neutralising or inhibiting composition can further comprises a component selected from the group consisting of pharmaceutically acceptable carriers, diluents and excipients.

Particular variants of this embodiments are an ADAMTS5 activity neutralising or inhibiting composition that comprises a peptide fragment of ADAMTS5 or of an ADAMTS5 activity neutralising or inhibiting composition that comprises a polyclonal antibody or of an ADAMTS5 activity neutralising or inhibiting composition that comprises a monoclonal antibody, or an antigen-binding fragment thereof, that binds to ADAMTS5 or of an ADAMTS5 activity neutralising or inhibiting composition that comprises antigen-binding fragment of a monoclonal antibody of the group consisting of a scFV, Fab, Fab2, F(ab′)2, Fv or dAB or of an ADAMTS5 activity neutralising or inhibiting composition that comprises a chimeric or humanized antibody or antigen-binding fragment thereof or of an ADAMTS5 activity neutralising or inhibiting composition that comprises the ADAMTS5 inhibitor (R)-N-((1-(4-(but-3-en-1-ylamino)-6-(((2-(thiophen-2-yl)thiazol-4-yl)methyl) amino)-1,3,5-triazin-2-yl)pyrrolidin-2-yl)methyl)-4-propylbenzenesulfonamide or 5-((1H-pyrazol-4-yl) methylene)-2-thioxothiazolidin-4-one for use in inducing brown fat (BAT) development in a mammalian subject for instance in a human, in impairing differentiation of adipocyte precursor cells into mature adipocytes (hyperplasia) in a mammalian subject for instance in a human, in preventing or reducing the de novo adipogenesis or de novo fat development and in decreasing in hepatosteatosis. This ADAMTS5 activity neutralising or inhibiting composition can further comprises a component selected from the group consisting of pharmaceutically acceptable carriers, diluents and excipients.

According to one embodiment the above described embodiment comprises further neutralising or inhibiting the total aggrecanase activity (ADAMTS4 and ADAMTS5) or reducing the amount of ADAMTS5 antigen and of ADAMTS4 antigen to treat the mammalian subjects in need thereof. Such neutralising or inhibiting of the aggrecanase activity of ADAMTS4 and ADAMTS5 or the reduction of the amount of ADAMTS5 antigen and of ADAMTS4 antigen can also be by molecules consisting of peptides and peptidomimetics, antibodies, small molecule inhibitors, double-stranded RNA, aptamers and ribozymes as a single active molecule or as a combination of active molecules.

In one embodiment, the above described neutralising or inhibiting the total aggrecanase activity involves an ADAMTS5 activity neutralising or inhibiting composition comprises the ADAMTS5 inhibitor of the group consisting of N-({(2R)-1-[4-(3-Buten-1-ylamino)-6-({[2-(2-thienyl)-1,3-thiazol-4-yl]methyl}amino)-1,3,5-triazin-2-yl]-2-pyrrolidinyl}-methyl)-4-(trifluoromethyl)benzenesulfonamide; N-({(2R)-1-[4-(3-Buten-1-ylamino)-6-({[2-(2-thienyl)-1,3-thiazol-4-yl]methyl}amino)-1,3,5-triazin-2-yl]-2-pyrrolidinyl}-methyl)-4-propylbenzenesulfonamide; N-({(2R)-1-[4-(3-Buten-1-ylamino)-6-({[2-(2-thienyl)-1,3-thiazol-4-yl]methyl}amino)-1,3,5-triazin-2-yl]-2-pyrrolidinyl}-methyl)-2-naphthalenesulfonamide; 3-Fluoro-4-methyl-N-((R)-1-(4-((2-(naphthalen-1-yl)ethyl)-amino)-6-(((S)-2-oxoazepan-3-yl)amino)-1,3,5-triazin-2-yl)-pyrrolidin-3-yl)benzenesulfonamide; N-((R)-1-(4-(Benzylamino)-6-(((S)-2-oxoazepan-3-yl)amino)-1,3,5-triazin-2-yl)pyrrolidin-3-yl)-3-fluoro-4-methylbenzenesulfonamide; N-({(2R)-1-[4-(Butylamino)-6-({[2-(2-thienyl)-1,3-thiazol-4-yl]-methyl}amino)-1,3,5-triazin-2-yl]-2-pyrrolidinyl}methyl)-4-(trifluoromethyl)benzenesulfonamide; N-({(2R)-1-[4-[(Cyclopropylmethyl)amino]-6-({[2-(2-thienyl)-1,3-thiazol-4-yl]methyl}amino)-1,3,5-triazin-2-yl]-2-pyrrolidinyl}methyl)-4-(trifluoromethyl)benzenesulfonamide; N-({(2R)-1-[4-(Methylamino)-6-({[2-(2-thienyl)-1,3-thiazol-4-yl]methyl}amino)-1,3,5-triazin-2-yl]-2-pyrrolidinyl}methyl)-4-(trifluoromethyl)benzenesulfonamide; N-({(2R)-1-[4-Oxo-6-({[2-(2-thienyl)-1,3-thiazol-4-yl]methyl}-amino)-1,4-dihydro-1,3,5-triazin-2-yl]-2-pyrrolidinyl}methyl)-4-(trifluoromethyl)benzenesulfonamide; N-({(2R)-1-[4-({[2-(2-Thienyl)-1,3-thiazol-4-yl]methyl}amino)-1,3,5-triazin-2-yl]-2-pyrrolidinyl}methyl)-4-(trifluoromethyl)-benzenesulfonamide; N-({(2R)-1-[4-{[(2-Phenyl-1,3-thiazol-4-yl)methyl]amino}-6-(propylamino)-1,3,5-triazin-2-yl]-2-pyrrolidinyl}methyl)-4-propylbenzenesulfonamide; N-{[(2R)-1-(4-[Methyl(propyl)amino]-6-{[(2-phenyl-1,3-thiazol-4-yl)methyl]amino}-1,3,5-triazin-2-yl)-2-pyrrolidinyl]-methyl}-4-propylbenzenesulfonamide; N-{[(2R)-1-(4-[(2-Methylpropypl)amino]-6-{[(2-phenyl-1,3-thiazol-4-yl)methyl]amino}-1,3,5-triazin-2-yl)-2-pyrrolidinyl]-methyl}-4-propylbenzenesulfonamide; N-{[(2R)-1-(4-[(2-Cyclopropylethyl)amino]-6-{[(2-phenyl-1,3-thiazol-4-yl)methyl]amino}-1,3,5-triazin-2-yl)-2-pyrrolidinyl]-methyl}-4-propylbenzenesulfonamide; N-{[(2R)-1-(4-(Cyclopropylamino)-6-{[(2-phenyl-1,3-thiazol-4-yl)methyl]amino }-1,3,5-triazin-2-yl)-2-pyrrolidinyl]methyl}-4-propylbenzenesulfonamide; N-{[(2R)-1-(4-(Cyclopentylamino)-6-{[(2-phenyl-1,3-thiazol-4-yl)methyl]amino}-1,3,5-triazin-2-yl)-2-pyrrolidinyl]methyl}-4-propylbenzenesulfonamide; N-{[(2R)-1-(4-[(2-Cyanoethyl)amino]-6-{[(2-phenyl-1,3-thiazol-4-yl)methyl]amino}-1,3,5-triazin-2-yl)-2-pyrrolidinyl]-methyl }-4-propylbenzenesulfonamide; N-{[(2R)-1-(4-[(2-Hydroxyethyl)amino]-6-{[(2-phenyl-1,3-thiazol-4-yl)methyl]amino}-1,3,5-triazin-2-yl)-2-pyrrolidinyl]-methyl}-4-propylbenzenesulfonamide; N-{[(2R)-1-(4-{[2-(Methyloxy)ethyl]amino}-6-{[(2-phenyl-1,3-thiazol-4-yl)methyl]amino 1-1,3,5-triazin-2-yl)-2-pyrrolidinyl]-methyl}-4-propylbenzenesulfonamide; N-[((2R)-1-{4-{[(2-Phenyl-1,3-thiazol-4-yl)methyl]amino}-6-[(tetrahydro-2H-pyran-4-ylmethyl)amino]-1,3,5-triazin-2-yl}-2-pyrrolidinyl)methyl]-4-propylbenzenesulfonamide; N-{[(2R)-1-(4-(3-Buten-1-ylamino)-6-{[(2-phenyl-1,3-thiazol-4-yl)methyl]amino}-1,3,5-triazin-2-yl)-2-pyrrolidinyl]methyl}-4-(trifluoromethyl)benzenesulfonamide; N-({(2R)-1-[4-[(4-Biphenylylmethyl)amino]-6-(3-buten-1-ylamino)-1,3,5-triazin-2-yl]-2-pyrrolidinyl}methyl)-4-(trifluoromethypbenzenesulfonamide; N-({(2R)-1-[4-[(3-Biphenylylmethyl)amino]-6-(3-buten-1-ylamino)-1,3,5-triazin-2-yl]-2-pyrrolidinyl}methyl)-4-(trifluoromethypbenzenesulfonamide; N-{[(2R)-1-(4-(3-Buten-1-ylamino)-6-{[(2-methyl-1,3-thiazol-4-yl)methyl]amino}-1,3,5-triazin-2-yl)-2-pyrrolidinyl]methyl}-4-(trifluoromethyl)benzenesulfonamide; N-[((2R)-1-4-(3-Buten-1-ylamino)-6-[(phenylmethyl)amino]-1,3,5-triazin-2-yl}-2-pyrrolidinyl)methyl]-4-(trifluoromethyl)-benzenesulfonamide; N-[((2R)-1-{4-(Butylamino)-6-[(3-thienylmethyl)amino]-1,3,5-triazin-2-yl}-2-pyrrolidinyl)methyl]-4-propylbenzenesulfonamide; N-{[(2R)-1-(4-(Butylamino)-6-{[(4-methyl-2-thienyl)methyl]-amino}-1,3,5-triazin-2-yl)-2-pyrrolidinyl]methyl}-4-propylbenzenesulfonamide; N-{[(2R)-1-(4-(Butylamino)-6-{[(4-cyano-2-thienyl)methyl]-amino}-1,3,5-triazin-2-yl)-2-pyrrolidinyl]methyl}-4-propylbenzenesulfonamide; N-{[(2R)-1-(4-(Butylamino)-6-{[(6-methyl-2-pyridinyl)-methyl]amino -1,3,5-triazin-2-yl)-2-pyrrolidinyl]methyl}-4-propylbenzenesulfonamide; N-{[(2R)-1-(4-(Butylamino)-6-[(3-methylphenyl)methyl]-amino}-1,3,5-triazin-2-yl)-2-pyrrolidinyl]methyl}-4-propylbenzenesulfonamide; N-{[(2R)-1-(4-(Butylamino)-6-{[(1-methyl-1H-imidazol-4yl)-methyl]amino}-1,3,5-triazin-2-yl)-2-pyrrolidinyl]methyl}-4-propylbenzenesulfonamide; N-{[(2R)-1-(4-(Butylamino)-6-{[(4-butylphenyl)methyl]-amino }-1,3,5-triazin-2-yl)-2-pyrrolidinyl]methyl}-4-propylbenzenesulfonamide; N-({(2R)-1-[4-(Butylamino)-6-(cyclopentylamino)-1,3,5-triazin-2-yl]-2-pyrrolidinyl}methyl)-4-propylbenzenesulfonamide; N-[((2R)-1-{4-(Butylamino)-6-[(cyclopropylmethyl)amino]-1,3,5-triazin-2-yl}-2-pyrrolidinyl)methyl]-4-propylbenzenesulfonamide; N-[((2R)-1-{4-(Butylamino)-6-[(1-methylethyl)amino]-1,3,5-triazin-2-yl}-2-pyrrolidinyl)methyl]-4-propylbenzenesulfonamide; N-{[(2S)-1-(4-(3-Buten-1-ylamino)-6-{[(2-phenyl-1,3-thiazol-4-yl)methyl]amino}-1,3,5-triazin-2-yl)-2-pyrrolidinyl]methyl }-4-(trifluoromethyl)benzenesulfonamide; N-{[(3S)-1-(4-(3-Buten-1-ylamino)-6-{[(2-phenyl-1,3-thiazol-4-yl)methyl]amino}-1,3,5-triazin-2-yl)-3-pyrrolidinyl]methyl}-4-(trifluoromethyl)benzenesulfonamide; N-{[(3R)-1-(4-(3-Buten-1-ylamino)-6-{[(2-phenyl-1,3-thiazol-4-yl)methyl]amino}-1,3,5-triazin-2-yl)-3-pyrrolidinyl]methyl}-4-(trifluoromethyl)benzenesulfonamide; N-{[(2R)-1-(4-(3-Buten-1-ylamino)-6-{[(2-phenyl-1,3-thiazol-4-yl)methyl]amino }-1,3,5-triazin-2-yl)-2-pyrrolidinyl]methyl}-2-(trifluoromethyl)benzenesulfonamide; N-{[(2R)-1-(4-(3-Buten-1-ylamino)-6-{[(2-phenyl-1,3-thiazol-4-yl)methyl]amino}-1,3,5-triazin-2-yl)-2-pyrrolidinyl]methyl}-2-(trifluoromethypbenzenesulfonamide; N-{[(2R)-1-(4-(3-Buten-1-ylamino)-6-{[(2-phenyl-1,3-thiazol-4-yl)methyl]amino }-1,3,5-triazin-2-yl)-2-pyrrolidinyl]methyl}-4-(methyloxy)benzenesulfonamide; N-{[(2R)-1-(4-(3-Buten-1-ylamino)-6-{[(2-phenyl-1,3-thiazol-4-yl)methyl]amino}-1,3,5-triazin-2-yl)-2-pyrrolidinyl]methyl}-benzenesulfonamide; N-{[(2R)-1-(4-(3-Buten-1-ylamino)-6-{[(2-phenyl-1,3-thiazol-4-yl)methyl]amino}-1,3,5-triazin-2-yl)-2-pyrrolidinyl]methyl }-cyclopropanesulfonamide; (R)-N-((1-(4-(Butylamino)-6-(((2-(thiophen-2-yl)thiazol-4-yl)-methyl)amino)-1,3,5-triazin-2-yl)pyrrolidin-2-yl)methyl)-3-ethoxybenzene sulfonamide; (R)-N-((1-(4-(Butylamino)-6-(((2-(thiophen-2-yl)thiazol-4-yl)methyl)amino)-1,3,5-triazin-2-yl)pyrrolidin-2-yl)methyl)-3-hydroxybenzenesulfonamide; N-({2R)-1-[4-(Butylamino)-6-({[2-(2-thienyl)-1,3-thiazol-4-yl]-methyl}amino)-1,3,5-triazin-2-yl]-2-pyrrolidinyl}methyl)-4-hydroxybenzenesulfonamide; N-({(2R)-1-[4-(Butylamino)-6-({[2-(2-thienyl)-1,3-thiazol-4-yl]-methyl}amino)-1,3,5-triazin-2-yl]-2-pyrrolidinyl}methyl)-4-chlorobenzenesulfonamide; N-({2R)-1-[4-(Butylamino)-6-({[2-(2-thienyl)-1,3-thiazol-4-yl]-methyl}amino)-1,3,5-triazin-2-yl]-2-pyrrolidinyl}methyl)-3-chlorobenzenesulfonamide; N-({2R)-1-[4-(Butylamino)-6-({[2-(2-thienyl)-1,3-thiazol-4-yl]-methyl}amino)-1,3,5-triazin-2-yl]-2-pyrrolidinyl}methyl)-3,4-dimethylbenzenesulfonamide; N-({2R)-1-[4-(Butylamino)-6-({[2-(2-thienyl)-1,3-thiazol-4-yl]-methyl}amino)-1,3,5-triazin-2-yl]-2-pyrrolidinyl}methyl)-4-biphenylsulfonamide; N-({(2R)-1-[4-(Butylamino)-6-({[2-(2-thienyl)-1,3-thiazol-4-yl]-methyl}-amino)-1,3,5-triazin-2-yl]-2-pyrrolidinyl}methyl)-4-(methylsulfonyl)benzenesulfonamide; N-({(2R)-1-[4-(Butylamino)-6-({[2-(2-thienyl)-1,3-thiazol-4-yl]-methyl}amino)-1,3,5-triazin-2-yl]-2-pyrrolidinyl}methyl)-4-cyanobenzenesulfonamide; N-({(2R)-1-[4-(Butylamino)-6-({[2-(2-thienyl)-1,3-thiazol-4-yl]-methyl}amino)-1,3,5-triazin-2-yl]-2-pyrrolidinyl}methyl)-3-cyanobenzenesulfonamide and N-({(2R)-1-[4-(Butylamino)-6-({[2-(2-thienyl)-1,3-thiazol-4-yl]-methyl}amino)-1,3,5-triazin-2-yl]-2-pyrrolidinyl}methyl)-4-fluorobenzenesulfonamide.

The object of the present invention is in an embodiment achieved by means of an ADAMTS5 inhibitor for use in a treatment to induce brown fat (BAT) development in a mammalian subject for instance a human, to induce conversion of white fat (WAT) into BAT, of treating to impair differentiation of adipocyte precursor cells into mature adipocytes (hyperplasia) in a mammalian subject for instance in a human, to prevent or reduce the de novo adipogenesis or de novo fat development in a mammalian subject for instance a human, to modulate thermogenesis, to treat obesity and/or one or more pathological conditions resulting thereof, to prevent an obesity related metabolic diseases, such as insulin resistance or to protect against liver triglyceride accumulation and/or liver steatosis. This object concerns treatment of prevention of obesity and related metabolic diseases, including type 2 diabetes, insulin resistance, atherosclerosis or lipid disorders or triglyceride accumulation and/or steatosis in the liver, nonalcoholic fatty liver disease (ICD10 (2015) K76.0)), lipid storage disorder (ICD10 (2015) E75.6), a disorders of lipoprotein metabolism or other lipidaemia (ICD10 (2015) E78). This object concerns also treatment for modulation of thermogenesis, the subject is in need of treatment for obesity and/or one or more pathological conditions resulting thereof, to improve the metabolic profile, to prevent an obesity related metabolic diseases, such as insulin resistance or the subject is in need of protection against triglyceride accumulation and liver steatosis. A suitable ADAMTS5 inhibitor for the object of this invention is an agent that reduces the amount of ADAMTS5 antigen, that reduces the expression of ADAMTS5 or that inhibits the activity of ADAMTS5 or an ADAMTS5 inhibitor that inhibits or neutralises the activity of ADAMTS5 or depletes or reduces the amount of ADAMTS5 antigen in said subject. In a particular embodiment the ADAMTS5 inhibitor is selected from the group consisting of an ADAMTS5 peptide, a peptidomimetic, an antibody, a double-stranded RNA, an aptamer, a small interfering RNA (siRNA) and a ribozyme. For use in the treatment these compounds can be comprised in a pharmaceutical composition in association with at least one pharmaceutically acceptable excipient

In aspect, the present invention provides an ADAMTS5 inhibitor for use in a treatment to induce brown fat (BAT) development in a mammalian subject for instance a human, to induce conversion of white fat (WAT) into BAT, of treating to impair differentiation of adipocyte precursor cells into mature adipocytes (hyperplasia) in a mammalian subject for instance in a human, to prevent or reduce the de novo adipogenesis or de novo fat development in a mammalian subject for instance a human, to modulate thermogenesis, to treat obesity and/or one or more pathological conditions resulting thereof, to prevent an obesity related metabolic diseases, such as insulin resistance or to protect against liver triglyceride accumulation and/or liver steatosis, whereby the ADAMTS5 inhibitor is a small molecule inhibitor.

In another aspect, the present invention provides an ADAMTS5 inhibitor for use in a treatment to induce brown fat (BAT) development in a mammalian subject for instance a human, to induce conversion of white fat (WAT) into BAT, of treating to impair differentiation of adipocyte precursor cells into mature adipocytes (hyperplasia) in a mammalian subject for instance in a human, to prevent or reduce the de novo adipogenesis or de novo fat development in a mammalian subject for instance a human, to modulate thermogenesis, to treat obesity and/or one or more pathological conditions resulting thereof, to prevent an obesity related metabolic diseases, such as insulin resistance or to protect against liver triglyceride accumulation and/or liver steatosis, whereby the inhibitor is selected from the group consisting of a specific polyclonal antibody, a monoclonal antibody, a full-length antibody, a binding fragments of an antibody and a surrogate of an antibody.

In yet another aspect, the present invention provides an ADAMTS5 inhibitor for use in a treatment to induce brown fat (BAT) development in a mammalian subject for instance a human, to induce conversion of white fat (WAT) into BAT, of treating to impair differentiation of adipocyte precursor cells into mature adipocytes (hyperplasia) in a mammalian subject for instance in a human, to prevent or reduce the de novo adipogenesis or de novo fat development in a mammalian subject for instance a human, to modulate thermogenesis, to treat obesity and/or one or more pathological conditions resulting thereof, to prevent an obesity related metabolic diseases, such as insulin resistance or to protect against liver triglyceride accumulation and/or liver steatosis, whereby the inhibitor is a binding fragments of an antibody or a surrogate of an antibody selected from the group consisting of aFab′, F(ab′)2, Fab, Fv, vIgG, scFv fragments and surrobodies, rIgG, disulfide-stabilized Fv antibodies (dsFv), diabodies, triabodies, and single domain antibodies, such as a camelized antibody or nanobody or humanised camel or shark antibody or nanobody.

In yet another aspect, the present invention provides an ADAMTS5 inhibitor for use in a treatment to induce brown fat (BAT) development in a mammalian subject for instance a human, to induce conversion of white fat (WAT) into BAT, of treating to impair differentiation of adipocyte precursor cells into mature adipocytes (hyperplasia) in a mammalian subject for instance in a human, to prevent or reduce the de novo adipogenesis or de novo fat development in a mammalian subject for instance a human, to modulate thermogenesis, to treat obesity and/or one or more pathological conditions resulting thereof, to prevent an obesity related metabolic diseases, such as insulin resistance or to protect against liver triglyceride accumulation and/or liver steatosis, whereby the ADAMTS5 inhibitor is a peptide fragment of ADAMTS5 or the ADAMTS5 inhibitor is a polyclonal antibody, that binds to ADAMTS5 or the ADAMTS5 inhibitor is a monoclonal antibody, or an antigen-binding fragment thereof, that binds to ADAMTS5 or the ADAMTS5 inhibitor is a ADAMTS5 antigen-binding fragment of a monoclonal antibody of the group consisting of a scFV, Fab, Fab2, F(ab′)2, Fv or dAB or the ADAMTS5 inhibitor is a chimeric or humanized antibody or antigen-binding fragment thereof.

In yet another aspect, the present invention provides an ADAMTS5 inhibitor for use in a treatment to induce brown fat (BAT) development in a mammalian subject for instance a human, to induce conversion of white fat (WAT) into BAT, of treating to impair differentiation of adipocyte precursor cells into mature adipocytes (hyperplasia) in a mammalian subject for instance in a human, to prevent or reduce the de novo adipogenesis or de novo fat development in a mammalian subject for instance a human, to modulate thermogenesis, to treat obesity and/or one or more pathological conditions resulting thereof, to prevent an obesity related metabolic diseases, such as insulin resistance or to protect against liver triglyceride accumulation and/or liver steatosis, the ADAMTS5 inhibitor is an antisense oligonucleotides, siRNAs, miRNAs and ribozymes against ADAMTS5 and/or ADAMTS4 and anti ADAMTS5 and/or ADAMTS4 antibody therapy is by gene therapy.

In yet another aspect, the present invention provides an ADAMTS5 inhibitor for use in a treatment to induce brown fat (BAT) development in a mammalian subject for instance a human, to induce conversion of white fat (WAT) into BAT, of treating to impair differentiation of adipocyte precursor cells into mature adipocytes (hyperplasia) in a mammalian subject for instance in a human, to prevent or reduce the de novo adipogenesis or de novo fat development in a mammalian subject for instance a human, to modulate thermogenesis, to treat obesity and/or one or more pathological conditions resulting thereof, to prevent an obesity related metabolic diseases, such as insulin resistance or to protect against liver triglyceride accumulation and/or liver steatosis, whereby the ADAMTS5 inhibitor is the small molecule (R)-N-((1-(4-(but-3-en-1-ylamino)-6-(((2-(thiophen-2-yl)thiazol-4-yl)methyl)amino)-1,3,5-triazin-2-yl)pyrrolidin-2-yl)methyl)-4-propylbenzenesulfonamide or 5-((1H-pyrazol-4-yl) methylene)-2-thioxothiazolidin-4-one.

In yet another aspect, the present invention provides an ADAMTS5 inhibitor for use in a treatment to induce brown fat (BAT) development in a mammalian subject for instance a human, to induce conversion of white fat (WAT) into BAT, of treating to impair differentiation of adipocyte precursor cells into mature adipocytes (hyperplasia) in a mammalian subject for instance in a human, to prevent or reduce the de novo adipogenesis or de novo fat development in a mammalian subject for instance a human, to modulate thermogenesis, to treat obesity and/or one or more pathological conditions resulting thereof to prevent an obesity related metabolic diseases, such as insulin resistance or to protect against liver triglyceride accumulation and/or liver steatosis, whereby the ADAMTS5 inhibitor is the small molecule 5′-Phenyl-3′H-spiro[indoline-3,2′-[1,3,4]thiadiazol]-2-one inhibitor of ADAMTS-5, a N-substituted 2-phenyl-1-sulfonylamino-cyclopropane carboxylates ADAMTS-5 inhibitor, a 5-((1H-Pyrazol-4-yl)methylene)-2-thioxothiazolidin-4-one inhibitor of ADAMTS-5 or an aryl thioxothiazolidinone inhibitor of ADAMTS-5.

In yet another aspect, the present invention provides an ADAMTS5 inhibitor for use in a treatment to induce brown fat (BAT) development in a mammalian subject for instance a human, to induce conversion of white fat (WAT) into BAT, of treating to impair differentiation of adipocyte precursor cells into mature adipocytes (hyperplasia) in a mammalian subject for instance in a human, to prevent or reduce the de novo adipogenesis or de novo fat development in a mammalian subject for instance a human, to modulate thermogenesis, to treat obesity and/or one or more pathological conditions resulting thereof, to prevent an obesity related metabolic diseases, such as insulin resistance or to protect against liver triglyceride accumulation and/or liver steatosis, whereby the ADAMTS5 inhibitor is the small molecule N-[(5-Chloro-8-hydroxy-7-quinolinyl)(3-nitrophenyl)methyl]-N2-methyl-N2-phenylglycinamide (alias: Acetamide, N-[(5-chloro-8-hydroxy-7-quinolinyl)(3-nitrophenyl)methyl]-2-(methylphenylamino)- or alias N-((8-Hydroxy-5-substituted-quinolin-7-yl)(phenyl)methyl)-2-phenyloxy/amino-acetamide) inhibitor of ADAMTS-5.

In yet another aspect, the present invention provides an ADAMTS5 inhibitor for use in a treatment to induce brown fat (BAT) development in a mammalian subject for instance a human, to induce conversion of white fat (WAT) into BAT, of treating to impair differentiation of adipocyte precursor cells into mature adipocytes (hyperplasia) in a mammalian subject for instance in a human, to modulate thermogenesis, to treat obesity and/or one or more pathological conditions resulting thereof, to prevent an obesity related metabolic diseases, such as insulin resistance or to protect against liver triglyceride accumulation and/or liver steatosis, whereby the ADAMTS5 inhibitor is the small molecule AGG-523 (alias PF-05212371; PF-5212371; WAY-266523) aggrecanase selective inhibitor.

In yet another aspect, the present invention provides an ADAMTS5 inhibitor for use in a treatment to induce brown fat (BAT) development in a mammalian subject for instance a human, to induce conversion of white fat (WAT) into BAT, of treating to impair differentiation of adipocyte precursor cells into mature adipocytes (hyperplasia) in a mammalian subject for instance in a human, to prevent or reduce the de novo adipogenesis or de novo fat development in a mammalian subject for instance a human, to modulate thermogenesis, to treat obesity and/or one or more pathological conditions resulting thereof, to prevent an obesity related metabolic diseases, such as insulin resistance or to protect against liver triglyceride accumulation and/or liver steatosis, whereby the ADAMTS5 inhibitor is the small molecule ADAMTS-5 inhibitor of the group consisting of N-((8-Hydroxy-5-substituted-quinolin-7-yl)(phenyl)methyl)-2-phenyloxy/amino-acetamide and N-substituted 2-phenyl-1-sulfonylamino-cyclopropane carboxylate.

In yet another aspect, the present invention provides an ADAMTS5 inhibitor for use in a treatment to induce brown fat (BAT) development in a mammalian subject for instance a human, to induce conversion of white fat (WAT) into BAT, of treating to impair differentiation of adipocyte precursor cells into mature adipocytes (hyperplasia) in a mammalian subject for instance in a human, to prevent or reduce the de novo adipogenesis or de novo fat development in a mammalian subject for instance a human, to modulate thermogenesis, to treat obesity and/or one or more pathological conditions resulting thereof, to prevent an obesity related metabolic diseases, such as insulin resistance or to protect against liver triglyceride accumulation and/or liver steatosis, whereby the ADAMTS5 inhibitor is the small molecule of the group consisting of N-({(2R)-1-[4-(3-Buten-1-ylamino)-6-({[2-(2-thienyl)-1,3-thiazol-4-yl]methyl}amino)-1,3,5-triazin-2-yl]-2-pyrrolidinyl}-methyl)-4-(trifluoromethyl)benzenesulfonamide; N-({(2R)-1-[4-(3-Buten-1-ylamino)-6-({[2-(2-thienyl)-1,3-thiazol-4-yl]methyl}amino)-1,3,5-triazin-2-yl]-2-pyrrolidinyl}-methyl)-4-propylbenzenesulfonamide; N-({(2R)-1-[4-(3-Buten-1-ylamino)-6-({[2-(2-thienyl)-1,3-thiazol-4-yl]methyl}amino)-1,3,5-triazin-2-yl]-2-pyrrolidinyl}-methyl)-2-naphthalenesulfonamide; 3-Fluoro-4-methyl-N-((R)-1-(4-((2-(naphthalen-1-yl)ethyl)-amino)-6-(((S)-2-oxoazepan-3-yl)amino)-1,3,5-triazin-2-yl)-pyrrolidin-3-yl)benzenesulfonamide; N-((R)-1-(4-(Benzylamino)-6-(((S)-2-oxoazepan-3-yl)amino)-1,3,5-triazin-2-yl)pyrrolidin-3-yl)-3-fluoro-4-methylbenzenesulfonamide; N-({(2R)-1-[4-(Butylamino)-6-({[2-(2-thienyl)-1,3-thiazol-4-yl]-methyl}amino)-1,3,5-triazin-2-yl]-2-pyrrolidinyl}methyl)-4-(trifluoromethyl)benzenesulfonamide; N-({(2R)-1-[4-[(Cyclopropylmethyl)amino]-6-({[2-(2-thienyl)-1,3-thiazol-4-yl]methyl}amino)-1,3,5-triazin-2-yl]-2-pyrrolidinyl}methyl)-4-(trifluoromethyl)benzenesulfonamide; N-({(2R)-1-[4-(Methylamino)-6-({[2-(2-thienyl)-1,3-thiazol-4-yl]methyl}amino)-1,3,5-triazin-2-yl]-2-pyrrolidinyl}methyl)-4-(trifluoromethyl)benzenesulfonamide; N-({(2R)-1-[4-Oxo-6-({[2-(2-thienyl)-1,3-thiazol-4-yl]methyl}-amino)-1,4-dihydro-1,3,5-triazin-2-yl]-2-pyrrolidinyl}methyl)-4-(trifluoromethyl)benzenesulfonamide; N-({(2R)-1-[4-({[2-(2-Thienyl)-1,3-thiazol-4-yl]methyl}amino)-1,3,5-triazin-2-yl]-2-pyrrolidinyl}methyl)-4-(trifluoromethyl)-benzenesulfonamide; N-({(2R)-1-[4-{[(2-Phenyl-1,3-thiazol-4-yl)methyl]amino}-6-(propylamino)-1,3,5-triazin-2-yl]-2-pyrrolidinyl}methyl)-4-propylbenzenesulfonamide; N-{[(2R)-1-(4-[Methyl(propyl)amino]-6-{[(2-phenyl-1,3-thiazol-4-yl)methyl]amino}-1,3,5-triazin-2-yl)-2-pyrrolidinyl]-methyl}-4-propylbenzenesulfonamide; N-{[(2R)-1-(4-[(2-Methylpropypl)amino]-6-{[(2-phenyl-1,3-thiazol-4-yl)methyl]amino}-1,3,5-triazin-2-yl)-2-pyrrolidinyl]-methyl}-4-propylbenzenesulfonamide; N-{[(2R)-1-(4-(2-Cyclopropylethyl)amino]-6-{[(2-phenyl-1,3-thiazol-4-yl)methyl]amino}-1,3,5-triazin-2-yl)-2-pyrrolidinyl]-methyl}-4-propylbenzenesulfonamide; N-{[(2R)-1-(4-(Cyclopropylamino)-6-{[(2-phenyl-1,3-thiazol-4-yl)methyl]amino }-1,3,5-triazin-2-yl)-2-pyrrolidinyl]methyl}-4-propylbenzenesulfonamide; N-{[(2R)-1-(4-(Cyclopentylamino)-6-{[(2-phenyl-1,3-thiazol-4-yl)methyl]amino}-1,3,5-triazin-2-yl)-2-pyrrolidinyl]methyl}-4-propylbenzenesulfonamide; N-{[(2R)-1-(4-[(2-Cyanoethyl)amino]-6-{[(2-phenyl-1,3-thiazol-4-yl)methyl]amino}-1,3,5-triazin-2-yl)-2-pyrrolidinyl]-methyl }-4-propylbenzenesulfonamide; N-{[(2R)-1-(4-[(2-Hydroxyethyl)amino]-6-{[(2-phenyl-1,3-thiazol-4-yl)methyl]amino}-1,3,5-triazin-2-yl)-2-pyrrolidinyl]-methyl}-4-propylbenzenesulfonamide; N-{[(2R)-1-(4-{[2-(Methyloxy)ethyl]amino}-6-{[(2-phenyl-1,3-thiazol-4-yl)methyl]amino 1-1,3,5-triazin-2-yl)-2-pyrrolidinyl]-methyl}-4-propylbenzenesulfonamide; N-[((2R)-1-{4-{[(2-Phenyl-1,3-thiazol-4-yl)methyl]amino}-6-[(tetrahydro-2H-pyran-4-ylmethyl)amino]-1,3,5-triazin-2-yl}-2-pyrrolidinyl)methyl]-4-propylbenzenesulfonamide; N-{[(2R)-1-(4-(3-Buten-1-ylamino)-6-{[(2-phenyl-1,3-thiazol-4-yl)methyl]amino}-1,3,5-triazin-2-yl)-2-pyrrolidinyl]methyl}-4-(trifluoromethyl)benzenesulfonamide; N-({(2R)-1-[4-[(4-Biphenylylmethyl)amino]-6-(3-buten-1-ylamino)-1,3,5-triazin-2-yl]-2-pyrrolidinyl}methyl)-4-(trifluoromethypbenzenesulfonamide; N-({(2R)-1-[4-[(3-Biphenylylmethyl)amino]-6-(3-buten-1-ylamino)-1,3,5-triazin-2-yl]-2-pyrrolidinyl}methyl)-4-(trifluoromethypbenzenesulfonamide; N-{[(2R)-1-(4-(3-Buten-1-ylamino)-6-{[(2-methyl-1,3-thiazol-4-yl)methyl]amino}-1,3,5-triazin-2-yl)-2-pyrrolidinyl]methyl}-4-(trifluoromethyl)benzenesulfonamide; N-[((2R)-1-4-(3-Buten-1-ylamino)-6-[(phenylmethyl)amino]-1,3,5-triazin-2-yl}-2-pyrrolidinyl)methyl]-4-(trifluoromethyl)-benzenesulfonamide; N-[((2R)-1-{4-(Butylamino)-6-[(3-thienylmethyl)amino]-1,3,5-triazin-2-yl}-2-pyrrolidinyl)methyl]-4-propylbenzenesulfonamide; N-{[(2R)-1-(4-(Butylamino)-6-{[(4-methyl-2-thienyl)methyl]-amino}-1,3,5-triazin-2-yl)-2-pyrrolidinyl]methyl}-4-propylbenzenesulfonamide; N-{[(2R)-1-(4-(Butylamino)-6-{[(4-cyano-2-thienyl)methyl]-amino}-1,3,5-triazin-2-yl)-2-pyrrolidinyl]methyl}-4-propylbenzenesulfonamide; N-{[(2R)-1-(4-(Butylamino)-6-{[(6-methyl-2-pyridinyl)-methyl]amino -1,3,5-triazin-2-yl)-2-pyrrolidinyl]methyl}-4-propylbenzenesulfonamide; N-{[(2R)-1-(4-(Butylamino)-6-[(3-methylphenyl)methyl]-amino}-1,3,5-triazin-2-yl)-2-pyrrolidinyl]methyl}-4-propylbenzenesulfonamide; N-{[(2R)-1-(4-(Butylamino)-6-{[(1-methyl-1H-imidazol-4yl)-methyl]amino}-1,3,5-triazin-2-yl)-2-pyrrolidinyl]methyl}-4-propylbenzenesulfonamide; N-{[(2R)-1-(4-(Butylamino)-6-{[(4-butylphenyl)methyl]-amino }-1,3,5-triazin-2-yl)-2-pyrrolidinyl]methyl}-4-propylbenzenesulfonamide; N-((2R)-1-[4-(Butylamino)-6-(cyclopentylamino)-1,3,5-triazin-2-yl]-2-pyrrolidinyl)methyl)-4-propylbenzenesulfonamide; N-[((2R)-1-{4-(Butylamino)-6-[(cyclopropylmethyl)amino]-1,3,5-triazin-2-yl}-2-pyrrolidinyl)methyl]-4-propylbenzenesulfonamide; N-[((2R)-1-4-(Butylamino)-6-[(1-methylethyl)amino]-1,3,5-triazin-2-yl}-2-pyrrolidinyl)methyl]-{4-propylbenzenesulfonamide; N-{[(2S)-1-(4-(3-Buten-1-ylamino)-6-{[(2-phenyl-1,3-thiazol-4-yl)methyl]amino}-1,3,5-triazin-2-yl)-2-pyrrolidinyl]methyl }-4-(trifluoromethyl)benzenesulfonamide; N-{[(3S)-1-(4-(3-Buten-1-ylamino)-6-{[(2-phenyl-1,3-thiazol-4-yl)methyl]amino }-1,3,5-triazin-2-yl)-3-pyrrolidinyl]methyl}-4-(trifluoromethyl)benzenesulfonamide; N-{[(3R)-1-(4-(3-Buten-1-ylamino)-6-{[(2-phenyl-1,3-thiazol-4-yl)methyl]amino}-1,3,5-triazin-2-yl)-3-pyrrolidinyl]methyl}-4-(trifluoromethyl)benzenesulfonamide; N-{[(2R)-1-(4-(3-Buten-1-ylamino)-6-{[(2-phenyl-1,3-thiazol-4-yl)methyl]amino }-1,3,5-triazin-2-yl)-2-pyrrolidinyl]methyl}-2-(trifluoromethyl)benzenesulfonamide; N-{[(2R)-1-(4-(3-Buten-1-ylamino)-6-{[(2-phenyl-1,3-thiazol-4-yl)methyl]amino }-1,3,5-triazin-2-yl)-2-pyrrolidinyl]methyl}-2-(trifluoromethypbenzenesulfonamide; N-{[(2R)-1-(4-(3-Buten-1-ylamino)-6-{[(2-phenyl-1,3-thiazol-4-yl)methyl]amino }-1,3,5-triazin-2-yl)-2-pyrrolidinyl]methyl}-4-(methyloxy)benzenesulfonamide; N-{[(2R)-1-(4-(3-Buten-1-ylamino)-6-{[(2-phenyl-1,3-thiazol-4-yl)methyl]amino}-1,3,5-triazin-2-yl)-2-pyrrolidinyl]methyl}-benzenesulfonamide; N-{[(2R)-1-(4-(3-Buten-1-ylamino)-6-{[(2-phenyl-1,3-thiazol-4-yl)methyl]amino}-1,3,5-triazin-2-yl)-2-pyrrolidinyl]methyl }-cyclopropanesulfonamide; (R)-N-((1-(4-(Butylamino)-6-(((2-(thiophen-2-yl)thiazol-4-yl)-methyl)amino)-1,3,5-triazin-2-yl)pyrrolidin-2-yl)methyl)-3-ethoxybenzene sulfonamide; (R)-N-((1-(4-(Butylamino)-6-(((2-(thiophen-2-yl)thiazol-4-yl)methyl)amino)-1,3,5-triazin-2-yl)pyrrolidin-2-yl)methyl)-3-hydroxybenzenesulfonamide; N-({2R)-1-[4-(Butylamino)-6-({[2-(2-thienyl)-1,3-thiazol-4-yl]-methyl}amino)-1,3,5-triazin-2-yl]-2-pyrrolidinyl}methyl)-4-hydroxybenzenesulfonamide; N-({(2R)-1-[4-(Butylamino)-6-({[2-(2-thienyl)-1,3-thiazol-4-yl]-methyl}amino)-1,3,5-triazin-2-yl]-2-pyrrolidinyl}methyl)-4-chlorobenzenesulfonamide; N-({2R)-1-[4-(Butylamino)-6-({[2-(2-thienyl)-1,3-thiazol-4-yl]-methyl}amino)-1,3,5-triazin-2-yl]-2-pyrrolidinyl}methyl)-3-chlorobenzenesulfonamide; N-({2R)-1-[4-(Butylamino)-6-({[2-(2-thienyl)-1,3-thiazol-4-yl]-methyl}amino)-1,3,5-triazin-2-yl]-2-pyrrolidinyl}methyl)-3,4-dimethylbenzenesulfonamide; N-({2R)-1-[4-(Butylamino)-6-({[2-(2-thienyl)-1,3-thiazol-4-yl]-methyl}amino)-1,3,5-triazin-2-yl]-2-pyrrolidinyl}methyl)-4-biphenylsulfonamide; N-({(2R)-1-[4-(Butylamino)-6-({[2-(2-thienyl)-1,3-thiazol-4-yl]-methyl}-amino)-1,3,5-triazin-2-yl]-2-pyrrolidinyl}methyl)-4-(methylsulfonyl)benzenesulfonamide; N-({(2R)-1-[4-(Butylamino)-6-({[2-(2-thienyl)-1,3-thiazol-4-yl]-methyl}amino)-1,3,5-triazin-2-yl]-2-pyrrolidinyl}methyl)-4-cyanobenzenesulfonamide; N-({(2R)-1-[4-(Butylamino)-6-({[2-(2-thienyl)-1,3-thiazol-4-yl]-methyl}amino)-1,3,5-triazin-2-yl]-2-pyrrolidinyl}methyl)-3-cyanobenzenesulfonamide and N-({(2R)-1-[4-(Butylamino)-6-({[2-(2-thienyl)-1,3-thiazol-4-yl]-methyl}amino)-1,3,5-triazin-2-yl]-2-pyrrolidinyl}methyl)-4-fluorobenzenesulfonamide.

According to one embodiment the ADAMTS5 inhibitor is used as the sole active ingredient in the treatment. According to one embodiment the ADAMTS5 activity neutralising or inhibiting composition further comprises a component selected from the group consisting of pharmaceutically acceptable carriers, diluents and excipients. According to yet one embodiment the ADAMTS5 inhibitor further comprises also neutralising or inhibiting the total aggrecanase activity (ADAMTS4/ADAMTS5 inhibitor) or is combined with an ADAMTS4 inhibitor and preferably comprised in a pharmaceutical composition in association with at least one pharmaceutically acceptable excipient. This ADAMTS4 inhibitor can be an agent that reduces the amount of ADAMTS4 antigen, that reduces the expression of ADAMTS4 or that inhibits the activity of ADAMTS4 and as for the ADAMTS5 inhibitor this ADAMTS4 inhibitor can be an ADAMTS4 antagonist selected of the group of a peptide, a peptidomimetic, antibodies, a small molecule inhibitors, a double-stranded RNA, an aptamers and a ribozyme.

Some embodiments of the invention are set forth in claim format directly below:
1) An ADAMTS5 inhibitor for use in a treatment of an ADAMTS5 associated disorder in a subject, wherein the ADAMTS5 associated disorder is an overweight disorder or a metabolic disorder which is obesity.
2) The ADAMTS5 inhibitor according to embodiment 1, wherein the ADAMTS5 associated disorder is an overweight disorder or a metabolic disorder which is one or more of obesity, type II diabetes, insulin resistance, disorder associated with insufficient insulin activity, disrupted thermogenesis in overweight, liver triglyceride accumulation, hepatosteatosis, fatty liver or non-alcoholic fatty liver disease.
3) The ADAMTS5 inhibitor according to any one of the embodiments 1 to 2, whereby the ADAMTS5 inhibitor is an antigen binding protein comprising at least one first immunoglobulin variable domain capable of binding to human ADAMTS5.
4) The ADAMTS5 inhibitor according to any one of the embodiments 1 to 2, whereby the inhibitor is selected from the group consisting of a specific polyclonal, a monoclonal antibody, a full-length antibody, a binding fragment of an antibody and a surrogate of an antibody capable of binding to human ADAMTS5
5) The ADAMTS5 inhibitor according to any one of the embodiments 1 to 2, whereby the inhibitor is a binding fragment of an antibody or a surrogate of an antibody selected from the group consisting of aFab′, F(ab′)2, Fab, Fv, vIgG, scFv fragments and surrobodies, rIgG, disulfide-stabilized Fv antibodies (dsFv), diabodies, triabodies, and single domain antibodies, such as a camelized antibody or nanobody or humanised camel or shark antibody or nanobody and capable of binding to human ADAMTS5.
6) The ADAMTS5 inhibitor according to any one of the embodiments 1 to 2, whereby the ADAMTS5 inhibitor is an ADAMTS5 antigen-binding fragment of a monoclonal antibody of the group consisting of a scFV, Fab, Fab2, F(ab′)2, Fv or dAB and capable of binding to human ADAMTS5.
7) The ADAMTS5 inhibitor according to any one of the embodiments 1 to 6, whereby the ADAMTS5 inhibitor is a chimeric or humanized antibody or antigen-binding fragment thereof and capable of binding to human ADAMTS5.
8) The ADAMTS5 inhibitor according to any one of the embodiments 1 to 7, whereby the ADAMTS5 inhibitor specifically binds to ADAMTS5.
9) The ADAMTS5 inhibitor according to any one of the embodiments 1 to 8, whereby the ADAMTS5 inhibitor specifically binds to human ADAMTS5.
10) The ADAMTS5 inhibitor according to any one of the embodiments 1 to 9, whereby inhibitor is a monoclonal antibody comprising a heavy chain comprising CDRH1, CDRH2 and CDRH3 and a light chain comprising CDRL1, CDRL2 and CDRL3, wherein the complementarity determining regions (CDRs) of the heavy chain are selected from the group of: a) CDRH1 having at least about 80% sequence identity to amino acid sequence DAWMD; b) CDRH2 having at least about 70% sequence identity to amino acid sequence EIRHKANDHAIFYXESVKG; and c) CDRH3 having at least about 70% sequence identity to amino acid sequence TYYYGSSYGYCDV or PFAY; and the complementarity determining regions of the light chain are selected from the group of: d) CDRL1 having at least about 70% sequence identity to amino acid sequence KASQSVGTTIV or RTSENIYSYLA; e) CDRL2 having at least about 70% sequence identity to amino acid sequence NAKTLAE or SASNRXT; and f) CDRL3 having at least about 70% sequence identity to amino acid sequence QQYSSYPFT or QHHYGTPWT.
11) The ADAMTS5 inhibitor according to any one of the embodiments 1 to 9, whereby inhibitor is a monoclonal antibody comprising a heavy chain comprising CDRH1, CDRH2 and CDRH3 and a light chain comprising CDR1, CDRL2 and CDRL3, wherein the complementarity determining regions (CDRs) of the heavy chain are selected from: (a) CDRH1 is amino acid sequence DAWMD; (b) CDRH2 is select from amino acid sequence EIRHKANDHAIFYAESVKG, EIRNKANNHARHYAESVKG, EIRHKANDYAIFYDESVKG, EIRHKANDHAIFYDESVKG, or DIRNTANNHATFYAESVKG, EIRHKANDHAIFYDESVKG; and (c) CDRH3 is TYYYGSSYGYCDV or PFAY; and the complementarity determining regions of the light chain are selected from: (d) CDRL1 is select from amino acid sequence KASQSVGTTIV, RTSENIYSYLA, or KASQNVGTAVV; (e) CDRL2 is select from amino acid sequence NAKTLAE, SASNRHT, SASTRYT, or SASNRYT; and (f) CDRL3 is select from amino acid sequence QQYSSYPFT, QHHYGTPWT, QQYVNYPFT, or QQYTSYPFT.
12) The ADAMTS5 inhibitor according to any one of the embodiments 1 to 9, whereby inhibitor is a monoclonal antibody comprising six CDRs wherein CDRH1 is DAWMD (SEQ ID NO:2), CDRH2 is EIRNKANNHARHYAESVKG (SEQ ID NO:13), and CDRH3 is TYYYGSSYGYCDV (SEQ ID NO:18) and CDRL1 is RTSENIYSYLA (SEQ ID NO:20), CDRL2 is NAKTLAE (SEQ ID NO:22) and CDRL3 is QHHYGTPWT (SEQ ID NO:27).
13) The ADAMTS5 inhibitor according to any one of the embodiments 1 to 9, whereby inhibitor is a monoclonal antibody comprising six CDRs wherein CDRH1 is DAWMD (SEQ ID NO:2), CDRH2 is EIRHKANDHAIFYDESVKG (SEQ ID NO:15), and CDRH3 is PFAY (SEQ ID NO:5) and CDRL1 is KASQSVGTTIV (SEQ ID NO:19), CDRL2 is SASNRHT (SEQ ID NO:23) and CDRL3 is QQYTSYPFT (SEQ ID NO:29).
14) The ADAMTS5 inhibitor according to any one of the embodiments 1 to 13, whereby the inhibitor is a nucleic acid molecule or such in a vector to express a therapeutic agent according to any one of the embodiments 1 to 13
15) The ADAMTS5 inhibitor according to any one of the embodiments 1 to 13, whereby the ADAMTS5 inhibitor comprises an antibody, or antigen-binding fragment thereof, that binds to ADAMTS5 with a dissociation constant (K D) of 150 pM or less, as determined by real-time biospecific interaction analysis (BIA) using surface plasmon resonance (SPR) technology, or with an IC50 of 100 pM or less.
16) The ADAMTS5 inhibitor according to any one of the embodiments 1 to 13, whereby the ADAMTS5 inhibitor comprises an antibody or antigen binding fragment thereof binds to a neutralizing epitope of human ADAMTS5 with an affinity of at least about 5×10⁴ liter/mole as measured by an association constant (Ka).
17) The ADAMTS5 inhibitor according to any one of the embodiments 1 to 16, whereby said treatment of the subject induces one or more primary efficacy endpoints or direct efficacy endpoints of a) increase in thermogenesis in brown adipose tissue, b) increase in insulin sensitivity of skeletal muscle, white adipose tissue, or liver; c) increase in glucose tolerance, d) increase in basal respiration, maximal respiration rate, or uncoupled respiration, e) increase in metabolic rate, f) decrease in hepatosteatosis or g) decrease in baseline in body weight.
18) The ADAMTS5 inhibitor according to any one of the embodiments 1 to 16, which can modulate one or more of the following biological activities: thermogenesis in adipose cells, differentiation of adipose cells, and insulin sensitivity of adipose cells.
19) The ADAMTS5 inhibitor according to any one of the embodiments 1 to 16, wherein said treatment prevents or reduces the de novo adipogenesis or prevents or reduces de novo fat development.
20) The ADAMTS5 inhibitor according to any one of the embodiments 1 to 16, wherein said treatment impaires differentiation of adipocyte precursor cells into mature adipocytes (hyperplasia) in a mammalian subject for instance a human.
21) The ADAMTS5 inhibitor according to any one of the embodiments 1 to 2 or 17 to 20, whereby the ADAMTS5 inhibitor is selected from the group consisting of a peptide, a peptidomimetic, an antibody, a double-stranded RNA, an aptamer, a small interfering RNA (siRNA), a peptide fragment of ADAMTS5 and a ribozyme
22) The ADAMTS5 inhibitor according to 21, whereby the peptide, peptidomimetic, antibody, double-stranded RNA, aptamer, small interfering RNA (siRNA), peptide fragment of ADAMTS5 or ribozyme therapy against ADAMTS5 is by gene therapy and in vivo expression in subject of the ADAMTS5 inhibitor.
23) The ADAMTS5 inhibitor according to any one of the embodiments 1 to 22, the ADAMTS5 inhibitor further comprises a component selected from the group consisting of pharmaceutically acceptable carriers, diluents and excipients.
24) A method of treating increased non-exercise activity thermogenesis and increased metabolic rate comprising administering to a human in need thereof a therapeutically effective amount of an ADAMTS5 inhibitor which can modulate one or more of the following biological activities: thermogenesis in adipose cells, differentiation of adipose cells, and insulin sensitivity of adipose cells.
25) A method of treating an overweight or obese subject, the method comprising: administering to the subject in need thereof, an ADAMTS5 inhibitor, effective to result in weight loss in the subject.
26) A method for increasing energy expenditure in a mammal comprising an ADAMTS5 inhibitor sufficient to activate differentiation of said cells into brown fact cells in the mammal, wherein ADAMTS5 inhibition is induced using an ADAMTS5 inhibitor according to any one of the previous embodiments 1 to 24 and wherein the differentiated brown fat cells promote energy expenditure thereby increasing energy expenditure in the mammal.
27) The method according to any one of the embodiment 24 to 26, wherein the subject is a human.
28) The method according to any one of the embodiment 24 to 26, wherein the subject is a cat or a dog.
29) The method according to any one of the embodiment 24 to 26, wherein the subject has a Body Mass Index of at least about 25 kg/m2.
30) The method according to any one of the embodiment 24 to 26, wherein the subject has a BMI of at least about 35 kg/m2 and an overweight- or obesity-related condition.
31) The method according to any one of the embodiment 24 to 30, but with a small molecule ADAMTS5 inhibitor as disclosed in this application.
32) The ADAMTS5 inhibitor according to any one of the embodiments 1 to 2 or 17 to 20, whereby the ADAMTS5 inhibitor is a ADAMTS5 inhibiting small molecule as disclosed in this application 33)

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION

### DETAILED DESCRIPTION OF EMBODIMENTS OF THE INVENTION

The present invention provides, as a valid strategy to prevent/treat obesity and related metabolic diseases, such as insulin resistance and to protect against triglyceride accumulation and liver steatosis upon high fat feeding, specific neutralization of ADAMTS5 activity or reduction of antigen concentration to promote BAT development and/or to promote conversion of WAT into BAT and/or to improve the metabolic profile. This is also achieved by inhibition or neutralization of total aggrecanase (ADAMTS4 and ADAMTS5). Neutralization of aggrecanase activity can be achieved by proteinase inhibitors, neutralizing antibodies (polyclonal, monoclonal, any antibody fragment . . . ), any genetic approach (knockdown, siRNA . . . ) or any other means resulting in reduction of ADAMTS4/ADAMTS5 antigen or activity. Polypeptides comprising at least one variable domain capable of binding and/or neutralising human ADAMTS5 are very suitable for the object of present invention. Antibodies or such neutralising polypeptides may be delivered by gene therapy. Therapeutic uses of brown and beige fat go beyond obesity and are a strategy of treatment for various metabolic disturbances, including type 2 diabetes, insulin resistance, atherosclerosis and lipid disorders.

Si/esiRNAs targeting ADAMTS5 suitable for the object of present invention are for instance nucleotides sequences of the group consisting of si/esiRNA with seq. ID 30, si/esiRNA with seq. ID 34, si/esiRNA with seq. ID 31, si/esiRNA with seq. ID 6, si/esiRNA with seq. ID 7, si/esiRNA with seq. ID 8, si/esiRNA with seq. ID 32 and si/esiRNA with seq. ID 33.

ADAMTS5 is for instance the human metalloprotease with the sequence as published on 15Mar. 2015, NCBI version NM_007038.3 GI:195539371, the sequence of Homo sapiens ADAM metallopeptidase with thrombospondin type 1 motif, 5 (ADAMTS5 ), mRNA (accession code NM_007038 & LOCUS NM_007038 9663 bp mRNA linear) or with the amino acid (AA) sequence of full human adamts5 or disintegrin and metalloproteinase with thrombospondin motifs 5 (ATS5_HUMAN, human ADAM-TS 5, ADAM-TS5, ADAMTS-5 or EC=3.4.24.)is of 930 AA as published by UniProtKB/Swiss-Prot on 1 Dec. and 11 Jan. 2011, sequence version 2 or 14 Oct. 2015 entry version 156). Such human ADAMTS5 (shown as SEQ ID NO:4) have been shown to cleave aggrecan between amino acids E373 and A374 producing the neoepitope ARGSVIL (SEQ ID NO:1). An inhibitor that reduces ADAMTS5 activity can for instance refer to a decrease in any and/or all of the activities associated with ADAMTS5 for instance such inhibitor that induces inhibition of Adamts5 induced ARGSVIL release from aggrecan. The amino Acid sequence of neoepitope ARGSVIL (SEQ ID NO:1) produced from aggrecan by cleavage of aggrecan between amino acids E373 and A374 by human ADAMTS5 is hereby provides as SEQ ID NO:1.

The terms “specific binding” or “specifically binds” refers to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the association constant KA is higher than 106 M-1. If necessary, nonspecific binding can be reduced without substantially affecting specific binding by varying the binding conditions. The appropriate binding conditions, such as concentration of antibodies, ionic strength of the solution, temperature, time allowed for binding, concentration of a blocking agent (e.g., serum albumin, milk casein), etc., may be optimized by a skilled artisan using routine techniques.

As used herein, the term “stringent” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. One example of stringent hybridization conditions is hybridization in 6*sodium chloride/sodium citrate (SSC) at about 45 deg. C., followed by at least one wash in 0.2*SSC, 0.1% SDS at 50 deg. C. A second example of stringent hybridization conditions is hybridization in 6*SSC at about 45 deg. C., followed by at least one wash in 0.2*SSC, 0.1% SDS at 55 deg. C. Another example of stringent hybridization conditions is hybridization in 6*SSC at about 45 deg. C., followed by at least one wash in 0.2*SSC, 0.1% SDS at 60 deg. C. A further example of stringent hybridization conditions is hybridization in 6*SSC at about 45 deg. C., followed by at least one wash in 0.2*SSC, 0.1% SDS at 65 deg. C. High stringent conditions include hybridization in 0.5M sodium phosphate, 7% SDS at 65 deg. C., followed by at least one wash at 0.2*SSC, 1% SDS at 65 deg. C.

The phrase “substantially as set out,” “substantially identical” or “substantially homologous” means that the relevant amino acid or nucleotide sequence (e.g., CDR(s), VH, or VL domain) will be identical to or have insubstantial differences (through conserved amino acid substitutions) in comparison to the sequences which are set out. Insubstantial differences include minor amino acid changes, such as 1 or 2 substitutions in a 5 amino acid sequence of a specified region. In the case of antibodies, the second antibody has the same specificity and has at least 50% of the affinity of the first antibody.

Sequences substantially identical or homologous (e.g., at least about 85% sequence identity) to the sequences disclosed herein are also part of this application. In some embodiment, the sequence identity can be about 85%, 90%, 95%, 96%, 97%, 98%, 99% or higher. Alternatively, substantial identity or homology exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

The term “therapeutic agent” is a substance that treats or assists in treating a medical disorder. Therapeutic agents may include, but are not limited to, substances that modulate immune cells or immune responses in a manner that complements the ADAMTS5 activity of anti-ADAMTS5 antibodies. Non-limiting examples and uses of therapeutic agents are described herein.

As used herein, a “therapeutically effective amount” of an anti-ADAMTS5 antibody refers to an amount of an antibody which is effective, upon single or multiple dose administration to a subject (such as a human patient) at treating, preventing, curing, delaying, reducing the severity of, and/or ameliorating at least one symptom of a disorder or recurring disorder, or prolonging the survival of the subject beyond that expected in the absence of such treatment.

The term “treatment” refers to a therapeutic or preventative measure. The treatment may be administered to a subject having a medical disorder or who ultimately may acquire the disorder, in order to prevent, cure, delay, reduce the severity of, and/or ameliorate one or more symptoms of a disorder or recurring disorder, or in order to prolong the survival of a subject beyond that expected in the absence of such treatment.

“Clinically meaningful endpoints ” directly measure how a patient feels, functions, or survives. They are the basis of basis for approval of new drugs. “Direct endpoints”, in contrast to surrogate endpoints, are endpoints that in themselves represent or characterize the clinical outcome of interest. They are basis for approval of new drugs (Eugene J. Sullivan, MD FCCP Chief Medical Officer United Therapeutics and Lung Rx U.S. Food and Drug Administration on Clinical Trial Endpoints)

An ADAMTS5 inhibitor reduces the amount of ADAMTS5 antigen, reduces the expression of ADAMTS5 and/or inhibits the activity of ADAMTS5. An ADAMTS4 inhibitor reduces the amount of ADAMTS4 antigen, reduces the expression of ADAMTS5 and/or inhibits the activity of ADAMTS4 or an ADAMTS5 inhibitor inhibits or neutralises the activity of ADAMTS5 or depletes ADAMTS5 antigen or reduces the amount of ADAMTS5 antigen. An ADAMTS5/ADAMTS4 inhibitor reduces the amount of ADAMTS5 antigen and ADAMTS4 antigen, reduces the expression of ADAMTS5 and ADAMTS4 and/or inhibits the activity of ADAMTS5 and ADAMTS4.

In work leading to the present invention the contribution of ADAMTS5 to adipose tissue development was evaluated using mice with specific gene inactivation (ADAMTS5^{-/-}. The data obtained point to a functional role of ADAMTS5 in BAT development, and establish the potential of ADAMTS5 inhibition/inhibitors (inhibition of ADAMTS5 activity and/or expression) to promote BAT development. These conclusions are supported by the findings that ADAMTS5^{-/-} mice kept on standard chow (SFD) or on high fat diet (HFD) have higher BAT mass and more browning of WAT as compared to wild-type littermates. Hence, in conclusion, the data surprisingly indicate that inhibition of only a single matrix metalloproteinase, i.e., ADAMTS5, is able to promote accumulation of BAT. Furthermore depletion of ADAMTS5 limits triglyceride accumulation (steatosis) in the liver of mice in vivo. In addition ADAMTS5^{-/-} mice kept on a sugar-rich diet (MCC) developed less WAT as compared to WT controls.

Moreover the contribution of ADAMTS5 to adipogenesis was evaluated using specific gene inactivation in 3T3-F442A pre-adipocytes as well as in mice. The data obtained point to a functional role of ADAMTS5 in adipogenesis, and establish the potential of ADAMTS5 inhibition/inhibitors (inhibition of ADAMTS5 activity and/or expression) to inhibit it. These conclusions are supported by the findings that ADAMTS5 knock-down in 3T3-F442A pre-adipocytes results in reduced differentiation in cell culture in vitro as well as in a mouse model of in vivo adipogenesis. Furthermore, embryonic fibroblasts derived from ADAMTS5 deficient mice show reduced differentiation into mature adipocytes. Hence, the data surprisingly indicate that inhibition of only a single matrix metalloproteinase, i.e., ADAMTS5, is able to impair adipogenesis in vitro and in vivo.

Obesity (according to the International Statistical Classification of Diseases and Related Health Problems (ICD-10 Version:2015) classification E66) includes anyone of the following disorders: obesity due to excess calories (E66.0), drug-induced obesity (E66.1), extreme obesity with alveolar hypoventilation (E66.2), morbid obesity (E66.8) or simple obesity NOS (E66.9). Adipogenesis in its broadest sense is the production and formation of fat or adipose tissue, i.e., loose connective tissue composed of adipocytes. Important locations of fat or adipose tissue include fat tissue beneath the skin (subcutaneous fat) and fat tissue surrounding internal organs (visceral or gonadal fat). Accumulation of excess body fat may lead to symptoms such as overweight (body mass index between 25 and 29.9 kg/m²), or worse, obesity (body mass index of 30 kg/m² or higher). Whereas WAT stores triglycerides as fat, BAT regulates dissipation of energy as heat, and is therefore suggested to be beneficial in preventing obesity (Ginter E, Simko V Brown fat tissue—a potential target to combat obesity. Bratisl Lek Listy. 2012; 113(1):52-6, Saito M Human brown adipose tissue: regulation and anti-obesity potential. Endocr J. 2014; 61(5):409-16, Loyd C, Obici S. Brown fat fuel use and regulation of energy homeostasis. Curr Opin Clin Nutr Metab Care. 2014; 17(4):368-72, Lidell M E, Betz M J, Enerback S. Brown adipose tissue and its therapeutic potential. J Intern Med. 2014; 276(4):364-77 and Langin D. Recruitment of brown fat and conversion of white into brown adipocytes: strategies to fight the metabolic complications of obesity? Biochim Biophys Acta. 2010; 1801(3):372-6).

An association exists between excessive body weight (overweight or obesity) and various diseases, in particular cardiovascular and cerebrovascular diseases, hypertension, diabetes mellitus type 2, sleep apnea, certain types of cancer, gallstones and osteoarthritis. As a result, excessive body weight reduces life expectancy. With rates of adult and childhood excessive body weight increasing, authorities view it as a serious public health problem. Furthermore, elevated triglyceride levels in the liver lead to steatosis.

The current invention relates to methods of reducing, controlling, treating, preventing or inhibiting the negative effects of fat-rich diets on the development of the above-described phenomena. In view of the unexpected fact that inhibition or deletion of a single matrix metalloproteinase (ADAMTS5) is able to promote BAT development and to limit triglyceride accumulation and impair formation of mature adipocytes, our invention comprises inhibiting the activity and/or the expression of ADAMTS5 or of total aggrecanases activity. More in particular, the activity and/or the expression of ADAMTS5 is selectively inhibited, meaning that the activity and/or the expression of other MMPs is not or not significantly inhibited. As way of example, the activity and/or expression of ADAMTS5 may be inhibited by (or by up to or at least) 50%, (or by up to or at least) 60%, 70%, 75%, 80%, 90% (or by up to or at least) 95% whereas the activity and/or the expression of any other individual MMP may remain unaffected (e.g., at least 95 to 100% active), may be slightly reduced (e.g., at least 75% active, at least 80% active, at least 85% active or at least 90% active), or may increase (e.g., at least 105% active, at least 110% active, at least 120% active) in which levels of activity or expression are expressed relative to the pre-treatment conditions.

Thus, in a first aspect, the invention relates to methods of promoting BAT development and prevention of excessive body weight in a mammal subjected to adipogenesis-inducing conditions, said methods comprising inhibiting the activity and/or expression of aggrecanases (ADAMTS5 and/or ADAMTS4) in said subject. By said inhibition the induction of BAT and/or reduction of triglyceride accumulation in said mammal is obtained.

In a further aspect, the invention covers methods for reducing the amount of existing adipose or fat tissue in mammals, i.e., adipose or fat tissue already grown or accumulated in a mammal, said method comprising inhibiting the activity and/or expression of ADAMTS5 in said subject. By said inhibition the reduction in the amount of existing adipose or fat tissue in said mammal is obtained.

Yet another aspect, the invention relates to methods of preventing differentiation of adipose precursor cells into mature adipocytes (hyperplasia) in a mammal subjected to adipogenesis-inducing conditions, said methods comprising inhibiting the activity and/or expression of aggrecanase (ADAMTS5) in said subject.

In any of the above methods said mammal can be a human.

The term “inhibition of expression” as used herein refers to the inhibition of gene transcription and/or translation of a gene transcript (mRNA), in particular of the ADAMTS5 gene or mRNA. Within the scope of the invention is included the well-known use of oligonucleotide anti-sense RNA and DNA molecules, small interfering RNAs (siRNAs) and ribozymes, that all function to inhibit the translation of ADAMTS5 mRNA into protein. Anti-sense RNA and DNA molecules act to directly block the translation of mRNA by binding to targeted mRNA and preventing translation into protein. Antisense oligonucleotides may be derived from the translation initiation site of the ADAMTS5 gene. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage of said target RNA. Within the scope of the invention are engineered ribozyme molecules (e.g. hammerhead motif ribozymes) that specifically and efficiently catalyze endonucleolytic cleavage of ADAMTS5 RNA sequences. Specific ribozyme cleavage sites within any potential RNA target are initially identified by scanning the target molecule for ribozyme cleavage sites. Once identified, short RNA sequences of between 15 and 20 ribonucleotides corresponding to the region of the target gene containing the cleavage site may be evaluated for predicted structural features such as secondary structure that may render the oligonucleotide sequence unsuitable. Both anti-sense RNA and DNA molecules and ribozymes of the invention may be prepared by any method known in the art for the synthesis of nucleic acid molecules. These include techniques for chemically synthesizing oligonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Short interference RNA molecules (siRNA) can be used for inhibiting expression of ADAMTS5. Such interference RNA molecules can be generated based on the sequence of the ADAMTS5 gene. RNA interference (RNAi) is based on the degradation of particular target sequences by the design of short interference RNA oligos (siRNA) which recognize the target sequence and subsequently trigger their degradation. In general siRNA duplexes are shorter than 30 nucleotides. MicroRNAs (miRNAs) are small single-stranded RNA molecules of about 19-25 nucleotides in length which can naturally direct gene silencing. They act via a mechanism sharing features with the RNAi pathway (Bartel D P. MicroRNAs: genomics, biogenesis, mechanism, and function. Cell. 2004; 116(2):281-97, Kim V N. MicroRNA biogenesis: coordinated cropping and dicing. Nat Rev Mol Cell Biol. 2005; 6(5):376-85 and Chen K, Rajewsky N. The evolution of gene regulation by transcription factors and microRNAs. Nat Rev Genet. 2007; 8(2):93-103). miRNAs can be generated and cloned into vectors to achieve constitutive expression in a target cell and resulting in shutting down expression of the targeted gene.

Antisense oligonucleotides, siRNAs, miRNAs and ribozymes may be delivered by gene therapy. Gene therapy means the treatment by the delivery of therapeutic nucleic acids to patient's cells. In gene therapy a gene is delivered to the targeted cells of a patient followed by effective production of the therapeutic gene in said cells. Gene therapy can be achieved via non-viral gene delivery and virus-mediated gene delivery. This is extensively reviewed in, e.g., the January issue of Br. Med. Bull. Vol. 51 (1995) and by Ledley (Ledley F D. Nonviral gene therapy: the promise of genes as pharmaceutical products. Human Gene Therapy. 1995; 6(9):1129-44). The preparation and gene therapy vectors for the intracellular expression of siRNA duplexes is disclosed e.g., in WO02/44321.

The term “inhibition of activity” as used herein refers to inhibition of the activity of the ADAMTS5 protein. This may be achieved e.g., by specific chemical inhibitors, provided these are sufficiently specific for ADAMTS5. To obtain the desired specificity, antibodies raised against ADAMTS5 are excellent candidates to be screened for inhibiting its activity. In particular monoclonal antibodies (raised in whatever species; and eventually humanized in case the species in not human) are of interest. Furthermore, RNA aptamers can be designed as therapeutic reagents for disrupting ADAMTS5 protein function. Selection of aptamers in vitro allows for the rapid isolation of extremely rare RNAs that have high specificity and affinity for specific proteins. Exemplary RNA aptamer development is described in U.S. Pat. No. 5,270,163, by Ellington et al., (Ellington A D, Szostak J W. In vitro selection of RNA molecules that bind specific ligands. Nature. 1990; 346, 818-22), and by Tuerk and Gold (12. Tuerk C, Gold L. Systematic evolution of ligands by exponential enrichment: RNA ligands to bacteriophage T4 DNA polymerase. Science. 1990; 249, 505-10). RNA aptamers can bind to the three dimensional surfaces of a protein and can moreover frequently discriminate between discrete functional sites of a protein (Gold L, Polisky B, Uhlenbeck 0, Yarus M Diversity of oligonucleotide functions. Annu Rev Biochem. 1995; 64:763-97). As therapeutic reagents, aptamers not only have the combined advantages of antibodies and small molecular mass drugs, in addition, they can be produced in vivo using gene therapy vectors. The basic methodologies for inhibiting expression of a gene and/or inhibiting activity of a gene product are well-know to the skilled person and can easily be tailored to target the ADAMTS5 gene and/or gene product.

Any suitable ADAMTS5 antagonist may be used according to the invention, for example peptides and peptidomimetics, antibodies, small molecule inhibitors, double-stranded RNA, aptamers and ribozymes. Preferred antagonists include peptide fragments of ADAMTS5, double-stranded RNA, aptamers and antibodies

As will be recognized by skilled artisans, anti-ADAMTS5 antibodies having specific binding properties, such as the ability to bind a specific epitope of interest, can be readily obtained using the various antigens and immunogens described herein and assessing their ability to compete for binding ADAMTS5 with a reference antibody of interest. Any of the anti-ADAMTS5 antibodies described herein can be utilized as a reference antibody in such a competition assay.

In conducting an antibody competition study between a reference anti-ADAMTS5 antibody and any test antibody (irrespective of species or isotype), one may first label the reference with a label detectable either directly, such as, for example, a radioisotope or fluorophore, or indirectly, such as, for example biotin (detectable via binding with fluorescently-labelled streptavidin) or an enzyme (detectable via an enzymatic reaction), to enable subsequent identification. In this case, a labelled reference anti-ADAMTS5 antibody (in fixed or increasing concentrations) is incubated with a known amount of ADAMTS5, forming a ADAMTS5:labeled anti-ADAMTS5 antibody complex. The unlabeled test antibody is then added to the complex. The intensity of the complexed label is measured. If the test antibody competes with the labeled reference anti-ADAMTS5 antibody for ADAMTS5 by binding to an overlapping epitope, the intensity of the complexed label will be decrease relative to a control experiment carried out in the absence of test antibody.

Numerous methods for carrying out binding competition assays are known and can be adapted to yield results comparable to the assay described above.

An antibody is considered to compete for binding ADAMTS5 with a reference anti- ADAMTS5 antibody, and thus considered to bind approximately the same or an overlapping epitope of ADAMTS5 as the reference anti-ADAMTS5 antibody, if it reduces binding of the reference anti-ADAMTS5 antibody to ADAMTS5 in a competitive binding assay, by at least 50%, at a test antibody concentration in the range of 0.01-100 μg/mL (e.g., 0.01 μg/mL, 0.08 μg/mL, 0.4 μg/mL, 2 μg/mL, 10 μg/mL, 50 μg/mL or 100 μg/mL or other concentration within the stated range), although higher levels of reduction, for example, 60%, 70%, 80%, 90% or even 100%, may be desirable.

Antibodies of the present disclosure can also be derivatized, covalently modified, or conjugated to other molecules to alter their properties or improve their function. For example, but not by way of limitation, derivatized antibodies include antibodies that have been modified, e.g., by glycosylation, fucosylation, acetylation, pegylation, phosphorylation, amidation, formylation, derivatization by known protecting/blocking groups, linkage to a cellular ligand or other protein, etc. Alternatively, specific amino acids in the variable or constant regions can be altered to change or improve function. In one non-limiting example, amino acid residues in the Fc region of an antibody may be altered to increase the serum half- life of the antibody by increasing its binding to FcRn.

Anti-ADAMTS5 monoclonal antibodies include antibodies labeled with a detectable moiety. Such a label can be conjugated directly or indirectly to an anti-ADAMTS5 monoclonal antibody of the disclosure. The label can itself be detectable (e.g., radioisotope labels, isotopic labels, or fluorescent labels) or, in the case of an enzymatic label, can catalyze chemical alteration of a substrate compound or composition which is detectable. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions.

Although the various anti-ADAMTS5 antibodies useful in the methods and kits described herein have been exemplified with full length antibodies, skilled artisans will appreciate that binding fragments, or surrogate antibodies designed or derived from full-length antibodies or binding fragments, may also be used. Suitable fragments, surrogates, etc., include, but are not limited to, Fab′, F(ab′)2, Fab, Fv, vIgG, scFv fragments and surrobodies, rIgG, disulfide-stabilized Fv antibodies (dsFv), diabodies, triabodies, and single domain antibodies, such as a camelized antibody or nanobody.

Antibodies of the present disclosure can be produced according to any way known to those of ordinarily skill in the art. In one non-limiting example, antibodies may be obtained from natural sources, including from any species capable of producing antibodies, such as antibodies derived from humans, simians, chicken, goats, rabbits, and rodents (e.g., rats, mice, and hamsters). Other species are also possible. Antibodies may also be generated and isolated from systems that utilize genetic engineering or recombinant DNA technology, such as, but not limited to, expression of recombinant antibodies in yeast cells, bacterial cells, and mammalian cells in culture, such as CHO cells. Antibodies may also be fully or partially synthetic.

Monoclonal antibodies (MAb) of the present disclosure are not limited to antibodies produced through hybridoma technology. A monoclonal antibody is derived from a single clone, including any eukaryotic, prokaryotic, or phage clone, by any means available or known in the art. Monoclonal antibodies can be prepared using a wide variety of techniques known in the art including the use of hybridoma, recombinant, and phage display technologies, or a combination thereof.

The basic methodologies for inhibiting expression of a gene and/or inhibiting activity of a gene product are well-know to the skilled person and can easily be tailored to target the ADAMTS5 gene and/or gene product.

Gene Therapy

A particular embodiment of present invention concerns antisense oligonucleotides, siRNAs, miRNAs and ribozymes against ADAMTS5 and/or ADAMTS4 and anti ADAMTS5 and/or ADAMTS4 antibody therapy may be delivered by gene therapy for use in a treatment of present invention.

Various vectors and gene-delivery techniques are available in the art such as γ-retrovirus- and lentivirus-derived vectors, adenovirus vectors, adeno-associated virus (AAV) vectors, herpes simplex virus (HSV-1)-based vectors and non-viral DNA vectors and various delivery systems such as macromolecular conjugates, auto-associative lipid-based systems, hydrodynamic-pressure-based and electrotransfer/electroporation whereby the gene delivery can be imaged. Textbooks and handbooks such as

Advanced textbook on Gene Transfer, Gene Therapy and Genetic Pharmacology: principles, Delivery and Pharmacological Biomedical Applications of Nucleotide Based Therapies (Imperial College Press Daniel Scherman editor 2014) provide enabling description thereon. A particular aspect of present invention is to provide a recombinant antibody expression construct comprising the recombinant antibody vector against ADAMTS5 and/or ADAMTS4 and said nucleotide sequence encoding the immunoglobulin variable region amino acid sequence. Recombinant antibody expression constructs expressing recombinant antibody vector against ADAMTS5 and/or ADAMTS4 may be introduced into host cells of a mammalian subject by “gene transfer” methods that are well known in the art. The invention also provides an isolated nucleotide sequence encoding the antibody against ADAMTS5 and/or ADAMTS4 for use in the treatment of present invention, optionally wherein the sequence is provided in an antibody expression vector, optionally in a host cell. This also provides a method of producing a human antibody, the method comprising replacing the non-human vertebrate constant regions of the antibody with human antibody constant regions. This may include recombinant antibody vector of the first aspect and one or more reagents for insertion of another nucleotide sequence encoding an immunoglobulin variable region amino acid sequence into the vector. The one or more reagents may include a restriction endonuclease. The method of treatment includes electroporation, DEAE-dextran transfection, calcium phosphate precipitation, cationic liposome-mediated transfection, heat shock and microparticle bombardment, although without limitation thereto. These gene transfer methods may be used to effect stable or transient expression of recombinant antibodies by host cells, as required. In a particular aspect, the invention provides a method of producing a recombinant antibody expression construct including the step of inserting another nucleotide sequence that encodes an immunoglobulin variable region amino acid sequence into the recombinant antibody expression vector of the first aspect.

The compound for use in present can be a small molecule ADAMTS5 inhibitor as a single active ingredient or in combination with a ADAMTS4 inhibitor. These compounds can conveniently be formulated in pharmaceutical compositions using conventional techniques and excipients such as those described in “Remington's Pharmaceutical Sciences Handbook” MACK Publishing, New York, 18th ed., 1990. The compounds of the invention can be administered by intravescical instillation, by intravenous injection, as a bolus, in dermatological preparations (creams, lotions, sprays and ointments), by inhalation as well as orally in the form of capsules, tablets, syrup, controlled-release formulations and the like. According to a preferred embodiment, also in combination with the preceding embodiments, the pharmaceutical formulations of the invention may be for intravescical, intravenous, topical and oral administration.

The pharmaceutical composition of the invention may additionally comprise a pharmaceutically acceptable carrier, diluent, and/or adjuvant.

Pharmaceutical Compositions and Methods of Administration

Certain embodiments of the invention include compositions comprising the disclosed antibodies. The compositions may be suitable for pharmaceutical use and administration to patients. The compositions comprise an antibody of the present invention and a pharmaceutical excipient. As used herein, “pharmaceutical excipient” includes solvents, dispersion media, coatings, antibacterial and antifungal agents, isotonic and absorption delaying agents, etc., that are compatible with pharmaceutical administration. Use of these agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. Pharmaceutical compositions may be topically or orally administered, or capable of transmission across mucous membranes. Examples of administration of a pharmaceutical composition include oral ingestion or inhalation.

Administration may also be intravenous, intraperitoneal, intramuscular, intracavity, subcutaneous, cutaneous, or transdermal.

Solutions or suspensions used for intradermal or subcutaneous application typically include at least one of the following components: a sterile diluent such as water, saline solution, fixed oils, polyethylene glycol, glycerine, propylene glycol, or other synthetic solvent; antibacterial agents such as benzyl alcohol or methyl parabens; antioxidants such as ascorbic acid or sodium bisulfate; chelating agents such as ethylenediaminetetraacetic acid (EDTA); buffers such as acetate, citrate, or phosphate; and tonicity agents such as sodium chloride or dextrose. The pH can be adjusted with acids or bases. Such preparations may be enclosed in ampoules, disposable syringes, or multiple dose vials.

Solutions or suspensions used for intravenous administration include a carrier such as physiological saline, bacteriostatic water, Cremophor EL (Registered Trademark) (BASF, Parsippany, N.J.), ethanol, or polyol. In all cases, the composition must be sterile and fluid for easy syringability. Proper fluidity can often be obtained using lecithin or surfactants. The composition must also be stable under the conditions of manufacture and storage. Prevention of microorganisms can be achieved with antibacterial and antifungal agents, e.g., parabens, chlorobutanol, phenol, ascorbic acid, thimerosal, etc. In many cases, isotonic agents (sugar), polyalcohols (mannitol and sorbitol), or sodium chloride may be included in the composition. Prolonged absorption of the composition can be accomplished by adding an agent which delays absorption, e.g., aluminum monostearate and gelatin.

Oral compositions include an inert diluent or edible carrier. The composition can be enclosed in gelatin or compressed into tablets. For the purpose of oral administration, the antibodies can be incorporated with excipients and placed in tablets, troches, or capsules. Pharmaceutically compatible binding agents or adjuvant materials can be included in the composition. The tablets, troches, and capsules, may contain (1) a binder such as microcrystalline cellulose, gum tragacanth or gelatin; (2) an excipient such as starch or lactose, (3) a disintegrating agent such as alginic acid, Primogel, or corn starch; (4) a lubricant such as magnesium stearate; (5) a glidant such as colloidal silicon dioxide; or (6) a sweetening agent or a flavoring agent.

The composition may also be administered by a transmucosal or transdermal route. For example, antibodies that comprise a Fc portion may be capable of crossing mucous membranes in the intestine, mouth, or lungs (via Fc receptors). Transmucosal administration can be accomplished through the use of lozenges, nasal sprays, inhalers, or suppositories. Transdermal administration can also be accomplished through the use of a composition containing ointments, salves, gels, or creams known in the art. For transmucosal or transdermal administration, penetrants appropriate to the barrier to be permeated are used. For administration by inhalation, the antibodies are delivered in an aerosol spray from a pressured container or dispenser, which contains a propellant (e.g., liquid or gas) or a nebulizer. In certain embodiments, the antibodies of this invention are prepared with carriers to protect the antibodies against rapid elimination from the body. Biodegradable polymers (e.g., ethylene vinyl acetate, polyanhydrides, polyglycolic acid, collagen, polyorthoesters, polylactic acid) are often used. Methods for the preparation of such formulations are known by those skilled in the art. Liposomal suspensions can be used as pharmaceutically acceptable carriers too. The liposomes can be prepared according to established methods known in the art (U.S. Pat. No. 4,522,811).

The antibodies or antibody compositions of the invention are administered in therapeutically effective amounts as described. Therapeutically effective amounts may vary with the subject's age, condition, sex, and severity of medical condition. Appropriate dosage may be determined by a physician based on clinical indications. The antibodies or compositions may be given as a bolus dose to maximize the circulating levels of antibodies for the greatest length of time. Continuous infusion may also be used after the bolus dose.

As used herein, the term “subject” is intended to include human and non-human animals. Subjects may include a human patient having a disorder characterized by cells that express ADAMTS5, e.g., a cancer cell or an immune cell. The term “non-human animals” of the invention includes all vertebrates, such as non-human primates, sheep, dogs, cows, chickens, amphibians, reptiles, etc.

Examples of dosage ranges that can be administered to a subject can be chosen from: 1 mu g/kg to 20 mg/kg, 1 mu g/kg to 10 mg/kg, 1 mu g/kg to 1 mg/kg, 10 mu g/kg to 1 mg/kg, 10 mu g/kg to 100 mu g/kg, 100 mu g/kg to 1 mg/kg, 250 mu g/kg to 2 mg/kg, 250 mu g/kg to 1 mg/kg, 500 mu g/kg to 2 mg/kg, 500 mu g/kg to 1 mg/kg, 1 mg/kg to 2 mg/kg, 1 mg/kg to 5 mg/kg, 5 mg/kg to 10 mg/kg, 10 mg/kg to 20 mg/kg, 15 mg/kg to 20 mg/kg, 10 mg/kg to 25 mg/kg, 15 mg/kg to 25 mg/kg, 20 mg/kg to 25 mg/kg, and mg/kg to 30 mg/kg (or higher). These dosages may be administered daily, weekly, biweekly, monthly, or less frequently, for example, biannually, depending on dosage, method of administration, disorder or symptom(s) to be treated, and individual subject characteristics. Dosages can also be administered via continuous infusion (such as through a pump). The administered dose may also depend on the route of administration. For example, subcutaneous administration may require a higher dosage than intravenous administration.

In certain circumstances, it may be advantageous to formulate compositions in dosage unit form for ease of administration and uniformity of dosage. Dosage unit form as used herein refers to physically discrete units suited for the patient. Each dosage unit contains a predetermined quantity of antibody calculated to produce a therapeutic effect in association with the carrier. The dosage unit depends on the characteristics of the antibodies and the particular therapeutic effect to be achieved.

Toxicity and therapeutic efficacy of the composition can be determined by standard pharmaceutical procedures in cell cultures or experimental animals, e.g., determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Antibodies that exhibit large therapeutic indices may be less toxic and/or more therapeutically effective.

The data obtained from the cell culture assays and animal studies can be used to formulate a dosage range in humans. The dosage of these compounds may lie within the range of circulating antibody concentrations in the blood, that includes an ED50 with little or no toxicity. The dosage may vary within this range depending upon the dosage composition form employed and the route of administration. For any antibody used in the present invention, the therapeutically effective dose can be estimated initially using cell culture assays. A dose may be formulated in animal models to achieve a circulating plasma concentration range that includes the IC50 (i.e., the concentration of antibody which achieves a half-maximal inhibition of symptoms).

Several ADAMTS5 small molecules can be used for present invention. In a particular embodiment the ADAMTS-5 inhibitor is a 5′-Phenyl-3′H-spiro [indoline-3,2′-[1,3,4]thiadiazol]-2-one inhibitor of ADAMTS-5, a N-substituted 2-phenyl-1-sulfonylamino-cyclopropane carboxylates ADAMTS-5 inhibitor, a 5-((1H-Pyrazol-4-yl)methylene)-2-thioxothiazolidin-4-one inhibitor of ADAMTS-5 or a N-[(5-Chloro-8-hydroxy-7-quinolinyl)(3-nitrophenyl)methyl]-N2-methyl-N2-phenylglycinamide (alias: Acetamide, N-[(5-chloro-8-hydroxy-7-quinolinyl)(3-nitrophenyl)methyl]-2-(methylphenylamino)- or alias N-((8-Hydroxy-5-substituted-quinolin-7-yl)(phenyemethyl)-2-phenyloxy/amino-acetamide) inhibitor of ADAMTS-5 or an aryl thioxothiazolidinone inhibitor of ADAMTS-5.

In yet another a particular embodiment the ADAMTS-5 inhibitor is AGG-523 (alias PF-05212371; PF-5212371; WAY-266523) aggrecanase selective inhibitors

In a particular embodiment of present invention the small molecule ADAMTS-5 inhibitor is the N-((8-Hydroxy-5-substituted-quinolin-7-yl)(phenyl)methyl)-2-phenyloxy/amino-acetamide inhibitor with the following structure.

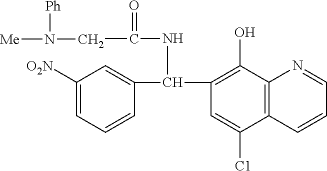

or it is the N-substituted 2-phenyl-1-sulfonylamino-cyclopropane carboxylates ADAMTS-5 inhibitor or is the a N-substituted 2-phenyl-1-sulfonylamino-cyclopropane carboxylate.

In yet another a particular embodiment the small molecule ADAMTS-5 inhibitor is a R1 and R2 substituted N-((8-hydroxy-5-substituted-quinolin-7-yl)(phenyl)methyl)-2-phenoxyacetamides of the general structure:

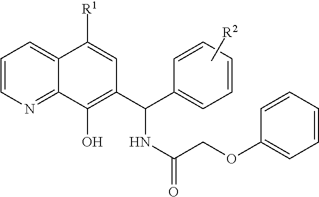

whereby R1 is one of the following H, F, NO2, Cl, Br, NO2, Me and R2 is one of the flowing H, 4-Me, 4-OMe, 4-CL, 4-NO2, 3-F, 3-NO2, 2-F, 2-Me, 2-CF3, 2-Cl, 2-NO2, and 3-NO2.

In yet another a particular embodiment the small molecule ADAMTS-5 inhibitor is a R2 and R3 substituted N-((8-hydroxy-5-substituted-quinolin-7-yl)(phenyl)methyl)-2-phenoxyacetamides of the general structure:

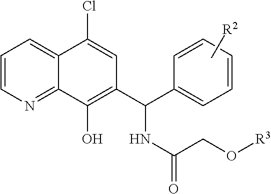

whereby R2 is one of the following 4-OMe, H, 3-F, 2-Cl and R3 is one of the following 3-Pyridyl, (4-Me)Ph, (4-OMe)Ph, (3-NMe2)Ph, (4-NHCOMe)Ph, (3-Me, 4-Cl)Ph, (4-OPh)Ph, (4-COMe)Ph, (3,5-Cl)Ph, (4-Cl)Ph, (4-tBu) Ph.

In yet another a particular embodiment the small molecule ADAMTS-5 inhibitor is a R1, R2 and R3 substituted N-((8-hydroxy-5-substituted-quinolin-7-yl)(phenyl)methyl)-2-phenoxyacetamides of the general structure:

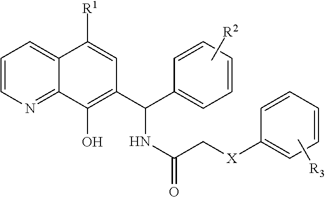

whereby R1 is one of the following H, F, NO2, Cl, Br, NO2, Me and R2 is one of the flowing H, 4-Me, 4-OMe, 4-CL, 4-NO2, 3-F, 3-NO2, 2-F, 2-Me, 2-CF3, 2-Cl, 2-NO2, 2 and 3-NO2 and R3 is one of the following 3-Pyridyl, (4-Me)Ph, (4-OMe)Ph, (3-NMe2)Ph, (4-NHCOMe)Ph, (3-Me, 4-Cl)Ph, (4-OPh)Ph, (4-COMe)Ph, (3,5-Cl)Ph, (4-Cl)Ph, (4-tBu) Ph.

In yet another a particular embodiment the small molecule ADAMTS-5 inhibitor is a N-substituted 2-phenyl-1-sulfonylamino-cyclopropane carboxylate with the general structure.

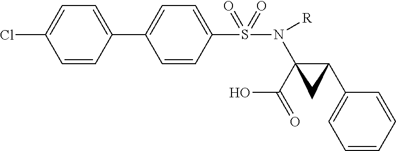

whereby the R group is one of the following structure.

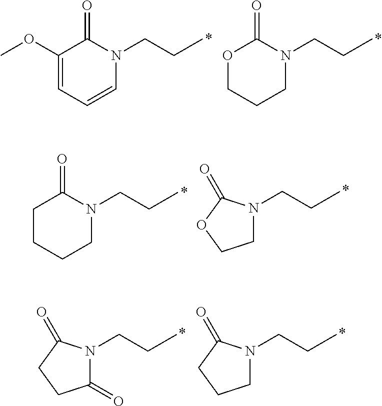

In yet another a particular embodiment the small molecule ADAMTS-5 inhibitor is a N-substituted 2-phenyl-1-sulfonylamino-cyclopropane carboxylate with the general structuretion.

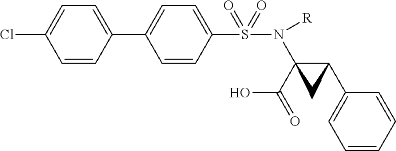

whereby the R group is

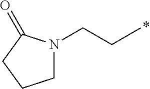

In yet a particular embodiment of present invention the small molecule ADAMTS-5 inhibitor is a N-substituted 2-phenyl-I -sulfonylamino-cyclopropane carboxylate with the general structuretion.

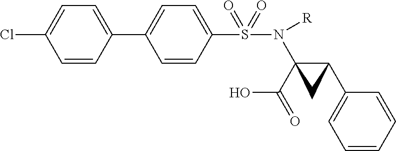

whereby the R group is

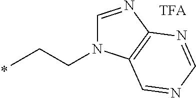

In yet a particular embodiment of present invention the small molecule ADAMTS-5 inhibitor is a N-substituted 2-phenyl-I -sulfonylamino-cyclopropane carboxylate with the general structuretion.

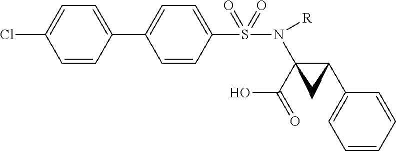

whereby the R group is one of the following structure.

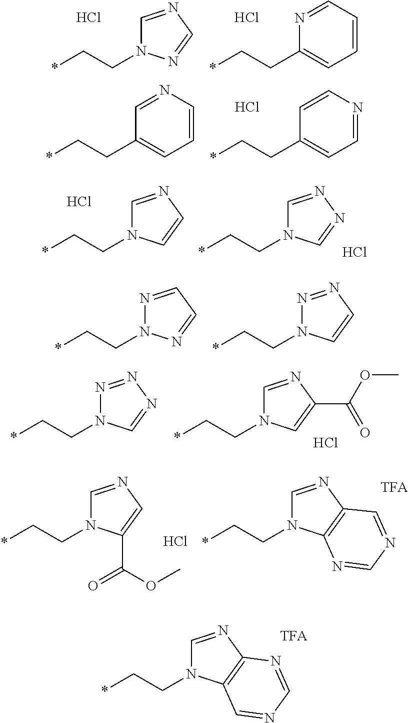

In yet a particular embodiment of present invention the small molecule ADAMTS-5 inhibitor is a 5′-Phenyl-3′H-spiro[indoline-3,2′-[1,3,4]thiadiazol]-2-one inhibitors of ADAMTS-5 (Aggrecanase-2)with the general structure.

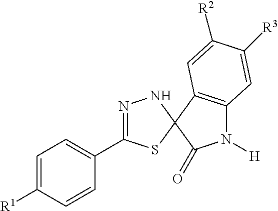

whereby R1 is one of the following H, Me, OMe and R2 is one of the flowing H, Br, Me, Et, OCF3, Cl, NO2, H and R3 is one of the following H, n-Pr, Me and Cl.

In yet a particular embodiment of present invention the small molecule ADAMTS-5 inhibitor is a 5i-Phenyl-3′H-spiro[indoline-3,2′-[1,3,4]thiadiazol]-2-one inhibitors of ADAMTS-5 (Aggrecanase-2)with the general structure.

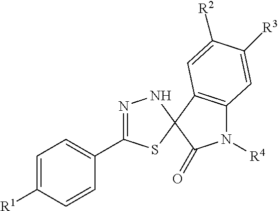

whereby R1 is one of the following H, Me, OMe and R2 is one of the flowing H, Me and R3 is one of the following H, n-Pr.

In yet a particular embodiment of present invention the small molecule ADAMTS-5 inhibitor is a 5′-Phenyl-3′H-spiro [indoline-3,2′-[1,3,4]thiadiazol]-2-one inhibitors of ADAMTS -5 (Aggrecanase-2)with the structure.

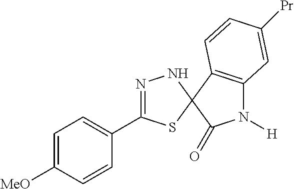

In yet a particular embodiment of present invention the small molecule ADAMTS-5 inhibitor is a 5′-Phenyl-3′H-spiro[indoline-3,2′-[1,3,4]thiadiazol]-2-one inhibitors of ADAMTS-5 (Aggrecanase-2)with the general structure.

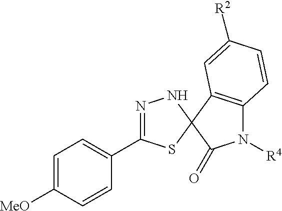

whereby R2 is H and R4 is Bn or whereby R2 is Me and R4 is Bn or whereby R2 is Me and R4 is o-Me Ph.

In yet a particular embodiment of present invention the small molecule ADAMTS-5 inhibitor is the N-substituted 2-[4-(4-Cl-phenyl)phenyl]sulfonylamino-3-phenyl-propionates inhibitor of ADAMTS-5 (Aggrecanase-2)with the structure.

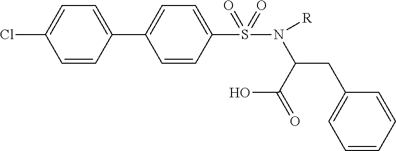

In yet a particular embodiment of present invention the small molecule ADAMTS-5 inhibitor is a benzimidazole class of tricyclic ring compound ADAMTS-5 (aggrecanase-2) inhibitor of the group consisting of ((E)-2-((4,5-dihydronaphtho[1,2-d]thiazol-2-yl)methylene)-thiazolidin-4-one and (E)-2-((4H-thiochromeno[4,3-d]thiazol-2-yl)methylene)-thiazolidin-4-one.

In yet a particular embodiment of present invention the small molecule ADAMTS-5 inhibitor is a benzimidazole class of tricyclic ring compound ADAMTS-5 (aggrecanase-2) inhibitor with the general structure

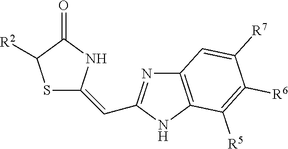

whereby R2 is −n—Pr, R5 is —H R6 is —H and R7 is —H.

In yet a particular embodiment of present invention the small molecule ADAMTS-5 inhibitor is a hADAMTS-5 inhibiting compound with the general structure

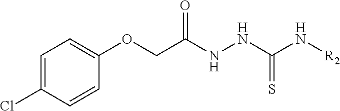

whereby R2 is −(4-Cyclohexyl)butyl (compound 1-(4-chlorophenoxyacethyl)-4-cyclohexylbutyl-thiosemicarbazide) or is —CH2-o-biphenyl (compound 1-(4-Chlorophenoxyacetyl)-4-(2-phenylbenzyl)-thiosemicarbazide).

An example of potent class of Adamts4 inhibtors useful for combining in an embodiment of present invention with a ADAMTS5 inhibitor are the ((4-keto)phenoxy)methylbiphenyl-4-sulfonamides for instance with the general structure

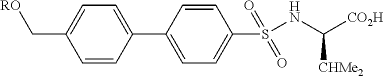

whereby R is of the group consisting of 4-(4-FC6H4CO)C6H4, 4-cyclohexylcarbonylphenyl, 4-Me2CHCOC6H4 and 1-oxo-1,2,3,4-tetrahydronaphthalen-6-yl.

### EXAMPLES

The following examples more fully illustrate preferred features of the invention, but are not intended to limit the invention in any way. All of the starting materials and reagents disclosed below are known to those skilled in the art, and are available commercially or can be prepared using well-known techniques.

### Example 1

1.1 Animals and Cells

Male athymic BALB/c NUDE mice were purchased from Charles River (Les Oncins, France). ADAMTS-5 wild-type (WT) and conditional knock-out (KO) mice were obtained as described elsewhere (Malfait A M, Ritchie J, Gil A S, Austin J S, Hartke J Qin W, et al. ADAMTS-5 deficient mice do not develop mechanical allodynia associated with osteoarthritis following medial meniscal destabilization. Osteoarthritis Cartilage. 2010; 18(4):572-80). Genotyping was performed using the forward 5′-TTTGAATTTGTCTTTGGAAGGCCTC-3′ and reverse 5′-TATCCCCGGATGAGTCAACACTGTC-3′ primer set. After a denaturation step at 94° C. for 2 min, isolated DNA was subjected to a polymerase chain reaction (PCR) consisting of denaturation at 94° C. for 30 sec, followed by 30 min of annealing at 63° C. and 1.5 min of elongation at 68° C. for 40 cycles.

3T3-F442A preadipocytes were obtained as described (Green H, Kehinde O. Spontaneous heritable changes leading to increased adipose conversion in 3T3 cells. Cell 1976; 7: 105-13).

1.2 Obesity Model

Five weeks old male mice were kept in microisolation cages on a 12h day/night cycle and fed at libitum with a high fat diet (HFD, Harlan Teklad TD 88137, Zeist, The Netherlands; 42% kcal as fat, 20.1 kJ g⁻¹) for 15 weeks (Lijnen H R, Maquoi E, Hansen L B, Van H B, Frederix L, Collen D. Matrix metalloproteinase inhibition impairs adipose tissue development in mice. Arterioscler Thromb Vasc Biol, 2002; 22, 374-9). In a separate study, WT and ADAMTS5^{-/-} were kept on a more sugar-rich diet for 4 weeks (Methioine/choline control diet, MCC; MP Biomedical, Brussels, Belgium; catalog number 960441), mice Weight and food intake were measured at weekly intervals. Mice were anesthetized by intraperitoneal injection of 60 mg kg⁻¹ Nembutal (Abbott Laboratories, North Chicago, Ill.). Blood was collected via the retroorbital sinus on trisodium citrate (final concentration 0.01 mol L⁻¹) and plasma was stored at −80° C. Intra-abdominal (gonadal, GON) and inguinal subcutaneous (SC) fat pads as well as BAT were removed and weighed; portions were snap-frozen in liquid nitrogen for histological and zymographic analysis, RNA or protein extraction. Other organs including kidneys, lungs, spleen, pancreas, liver and heart were also removed, weighed and snap-frozen. For comparison, mice of each genotype were kept on normal chow (SFD, KM-04-k12, Muracon, Carfil, Oud-Turnhout, Belgium; 13% kcal as fat, 10.9 kJ g⁻¹). Body weight and food intake were monitored weekly. Body temperature was measured using a rectal probe (TR-100, Fine Science Tools, Foster City, Calif.).

Livers were isolated for quantitation of liver steatosis by haematoxylin/eosin staining and extracts were prepared to determine triglyceride levels.

1.3 Cold Exposure Model

Eight weeks old male WT or ADAMTS5^{-/-} mice were kept at 24° C. (n=3 each) or at 4° C. to 6° C. (n=6 each) for 2 weeks and fed SFD (Rosell M, Kaforou M Frontini A, Okolo A, Chan Y W, Nikolopoulou E, Millership S, Fenech M E, MacIntyre D, Turner J O, Moore J D, Blackburn E, Gullick W J, Cinti S, Montana G, Parker M G, Christian M Brown and white adipose tissues: intrinsic differences in gene expression and response to cold exposure in mice. Am J Physiol Endocrinol Metab. 2014; 306(8):E945-64).

All animal experiments were approved by the local ethical committee (K U Leuven) and were performed in accordance with the guiding principles of the American Physiological Society.

1.4 Gene Expression Analysis

DNA-free total RNA was extracted BAT or WAT using the RNAeasy kit (Qiagen, Basel, Switzerland) according to the manufacturer's instructions. RNA concentrations were measured spectrophotometrically and total RNA samples were stored at −80° C. Complementary DNA was prepared from total RNA using the TaqMan® Reverse Transcription Reagents (Applied Biosystems, Foster City, Calif.). PCR reactions were performed from 10 ng/μl total RNA at 25° C. for 10 min, followed by amplification at 48° C. for 1 h and finally 5 min at 95° C. Quantitative real time PCR was performed in the ABI 7500 Fast Sequence detector using the TaqMan® Fast Universal PCR Master Mix (Applied Biosystems). The expression of markers of BAT (UCP-1, UCP-2, Cidea, PGC1a, PRDM16) was determined with appropriate primers and 6-carboxy-fluorescein (FAM) labeled probes. Expression levels were determined using gene expression assays from Applied Biosystems (Foster City, USA). Fold differences in gene expression were calculated with the ΔΔCt method, using β-actin as housekeeping gene. For the in vitro cell culture experiments, control cells at experimental day 0 were used as calibrator.

1.5 Statistical Analysis

Data are expressed as mean ±SEM. Differences between groups were analyzed with the non-parametric t test (Mann-Whitney, U-test). Statistical significance was set at p<0.05.

### Example 2

2.1 In vitro Preadipocyte Differentiation and ADAMTS5 Knock-Down

Murine 3T3-F442A preadipocytes were routinely grown at subconfluence in basal medium (Dulbecco's modified Eagle's medium (DMEM; Invitrogen, Paisley, UK) supplemented with 10% bovine calf serum (BCS, iron supplemented; Hyclone, Logan, Utah, USA) and 1% PenStrep (Invitrogen)). Cells were passaged when preconfluent. To induce differentiation (Van Hul M, Bauters D, Himmelreich U, Kindt N, Noppen B, Vanhove M et al. Effect of gelatinase inhibition on adipogenesis and adipose tissue development. Clin Exp Pharmacol Physiol. 2012; 39(1):49-56), cells were seeded at a density of 30×10³ cells cm-2 and grown to confluency (designated as ‘day 0’) in basal medium in an atmosphere of 95% humified air—5% CO2 at 37° C. After 2 days, cells were induced to differentiate for 48 h with induction medium (DMEM supplemented with 10% Fetal Bovine Serum (FBS), 17 nM insulin, 2 nM tri-iodothyronine (T3), 100 nM dexamethasone (DEX) and 100 μM methylisobutylxanthine (IBMX)). Thereafter, cultures were switched to differentiation medium (DMEM with 10% FBS, 17 μM insulin and 2 nM T3). The medium was replaced with fresh culture medium every 2 days. At regular time points during the differentiation of the cells, cell lysates were taken for RNA extraction. On experimental day 12, the extent of differentiation was assessed by quantification of Oil Red O uptake by lipid-containing cells. Cells were washed with phosphate-buffered saline (PBS), fixed in 1.5% glutaraldehyde in PBS for 5 min, stained for 3 h with a 0.2% Oil Red O solution (Sigma-Aldrich, Bornem, Belgium), washed and kept in tissue culture water. The stained fat droplets in the monolayer cells were visualized by light microscopy and photographed. For spectrophotometric quantification of lipid accumulation, the Oil Red O dye was extracted with isopropanol and the absorbance of the solution was read at 490 nm on an EL808 plate reader using KC4 DATA ANALYSIS software (Bio-tek Instruments, Winooski, Vt., USA). Alternatively, the stained area was determined by image analysis and expressed a percent of the total section area.

To obtain stable gene silencing of ADAMTS-5 (NM_011782) in 3T3-F442A preadipocytes, the ‘MISSION shRNA lentiviral transduction particles’ system (Sigma-Aldrich) was used. Several different target clones were provided ( )and MISSION non-target shRNA control transduction particles (SHC002V, hereafter ‘2V’) were used as negative control. Transduction efficiency was further enhanced with the ‘ExpressMag®’ transduction system (Sigma Aldrich). Briefly, for each lentiviral construct, triplicates of 40×10³ 3T3-F442A preadipocytes were seeded in a 6-well plate in basal medium. The next day, a mixture of 20 μl lentiviral particles and 30 μl beads was added to the cells in fresh basal medium without antibiotics. After 24 h, the medium was replaced by fresh basal medium containing a lethal dose of 2 μg/ml puromycin. Medium with puromycin was refreshed every two days until puromycin-resistant cells appeared. The puromycin-resistant clones were further expanded and assayed for knock-down of the target gene. Cells transduced with the negative control 2V were subjected to the same selection procedure. ADAMTS-5 antigen levels were determined by a commercially available ELISA (Cusabio).

2.2 In vitro Murine Embryonic Fibroblast Differentiation

Murine embryonic fibroblasts (MEFs) were isolated from 13.5 day old embryos obtained from ADAMTS-5 (ADAMTS-5^{-/-}) and wild-type ADAMTS-5n mice, and were grown in basal medium (Lijnen H R, Christiaens V, Scroyen L. Growth arrest-specific protein 6 receptor antagonism impairs adipocyte differentiation and adipose tissue development in mice. J Pharmacol Exp Ther. 2011; 337(2):457-64). Embryos were surgically removed and separated from maternal tissues and yolk sac. Heads were removed, bodies were minced into small pieces and were then incubated in a solution of DMEM and trypsin at 4° C. for 90 min, followed by 12 min at 37° C. The resulting pellet was resuspended in culture medium and cells were grown in basal medium. To induce differentiation (Dani C, Smith A G, Dessolin S, Leroy P, Staccini L, Villageois P, Darimont C, Ailhaud G. Differentiation of embryonic stem cells into adipocytes in vitro. J Cell Sci. 1997; 110:1279-85), cells were seeded at a density of 50×10³ cells cm-2 and grown to confluency in basal medium. Medium was refreshed when cells reached confluency, followed by a two day induction period with DMEM GlutaMAX supplemented with 10% FBS, 1.7 μM insulin, 1 μM DEX, 0.5 mM IBMX and 5 μM rosiglitazone. Cells were differentiated in DMEM GlutaMAX containing 10% FBS, 0.85 μM insulin and 5 μM rosiglitazone for 6 days. During the period of differentiation, cell lysates were collected for RNA and protein extraction. Lipids were stained with Oil Red O to monitor the degree of differentiation, as described above. Gene expression of adipogenic markers was monitored as described below.

2.3 De novo adipogenesis in vivo

To induce de novo fat pad information, 10⁷ 3T3-F442A preadipocytes (with or without ADAMTS5 knock-down), grown to near confluency and resuspended in 200 μl PBS, were injected subcutaneously in the back of 6 to 8 week old male athymic Balb/c NUDE mice (Scroyen I, Cosemans L, Lijnen H R. Effect of tissue inhibitor of matrix metalloproteinases-1 on in vitro and in vivo adipocyte differentiation. Thromb Res. 2009; 124(5):578-83, Mandrup S, Loftus T M, MacDougald O A, Kuhajda F P, Lane M D. Obese gene expression at in vivo levels by fat pads derived from s.c. implanted 3T3-F442A preadipocytes. Proc Natl Acad Sci USA. 1997; 94: 4300-5 and Neels J G, Thinnes T, Loskutoff D J. Angiogenesis in an in vivo model of adipose tissue development. Faseb J 2004; 18:983-5).

Mice were kept in microisolation cages on a 12 h day-night cycle and fed water and a HFD (TD88137, Harlan, Madison, Wis.; containing 42% kcal as fat, with a caloric value of 20.1 kJ per g) for 4 weeks. At the end of the experiment, mice were killed by i.p. injection of 60 mg/kg sodium pentobarbital (Nembutal, Abbott Laboratories, North Chicago, Ill.). Intra-abdominal gonadal (GON), inguinal subcutaneous (SC) fat tissue and de novo formed fat pads were removed and weighed.

All animal experiments were approved by the local ethical commmitte (K U Leuven) and performed in accordance with the NIH Guide for the Care and Use of Laboratory Animals (1996).

2.4 Gene Expression Analysis

DNA-free total RNA was extracted from 3T3-F442A cells or de novo formed fat using the RNAeasy kit (Qiagen, Basel, Switzerland) according to the manufacturer's instructions. RNA concentrations were measured spectrophotometrically and total RNA samples were stored at −80° C. Complementary DNA was prepared from total RNA using the TaqMan® Reverse Transcription Reagents (Applied Biosystems, Foster City, Calif.). PCR reactions were performed from 10 ng/μl total RNA at 25° C. for 10 min, followed by amplification at 48° C. for 1 h and finally 5 min at 95° C. Quantitative real time PCR was performed in the ABI 7500 Fast Sequence detector using the TaqMan® Fast Universal PCR Master Mix (Applied Biosystems). The expression of adipocyte markers (adipocyte fatty acid-binding protein (aP2; also known as fabp4), peroxisome proliferator-activated receptor-γ (PPARγ) and preadipocyte factor 1 (Pref-1)) was determined with appropriate primers and 6-carboxy-fluorescein (FAM) labeled probes. Expression levels were determined using gene expression assays from Applied Biosystems (Foster City, USA). Fold differences in gene expression were calculated with the ΔΔCt method, using β-actin as housekeeping gene. For the in vitro cell culture experiments, control cells at experimental day 0 were used as calibrator.

2.5 Statistical Analysis

Data are expressed as mean ±SEM. Differences were analyzed with the non-parametric t test (Mann-Whitney, U-test). Statistical significance was set at p<0.05.

### Example 3

### Effect of ADAMTS5 on Differentiation of Preadipocytes

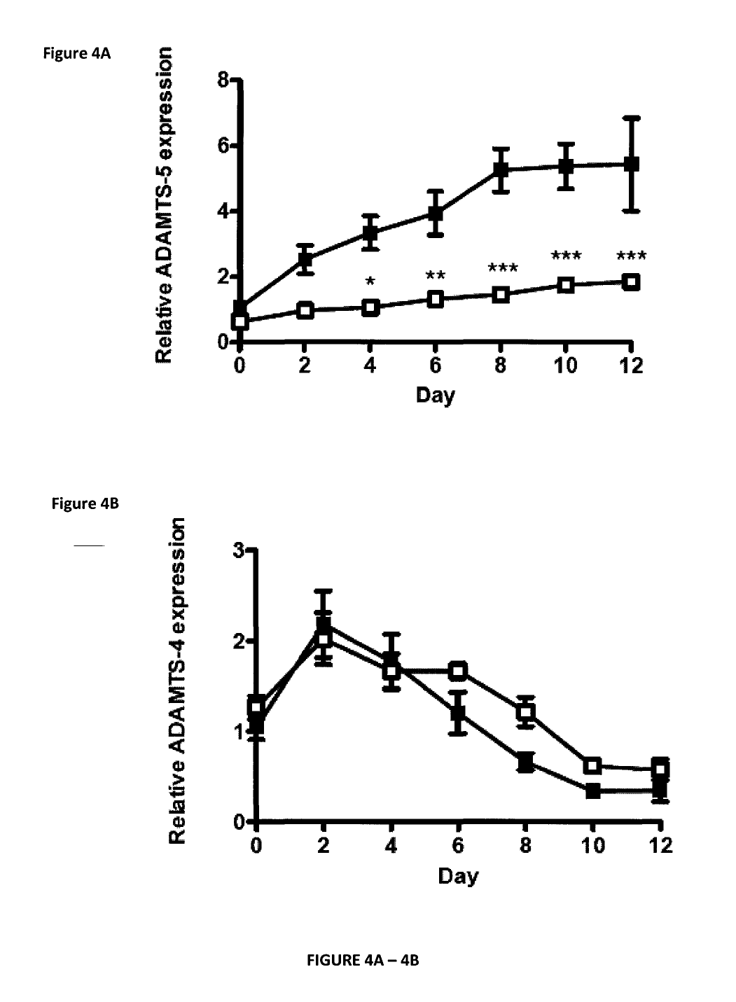

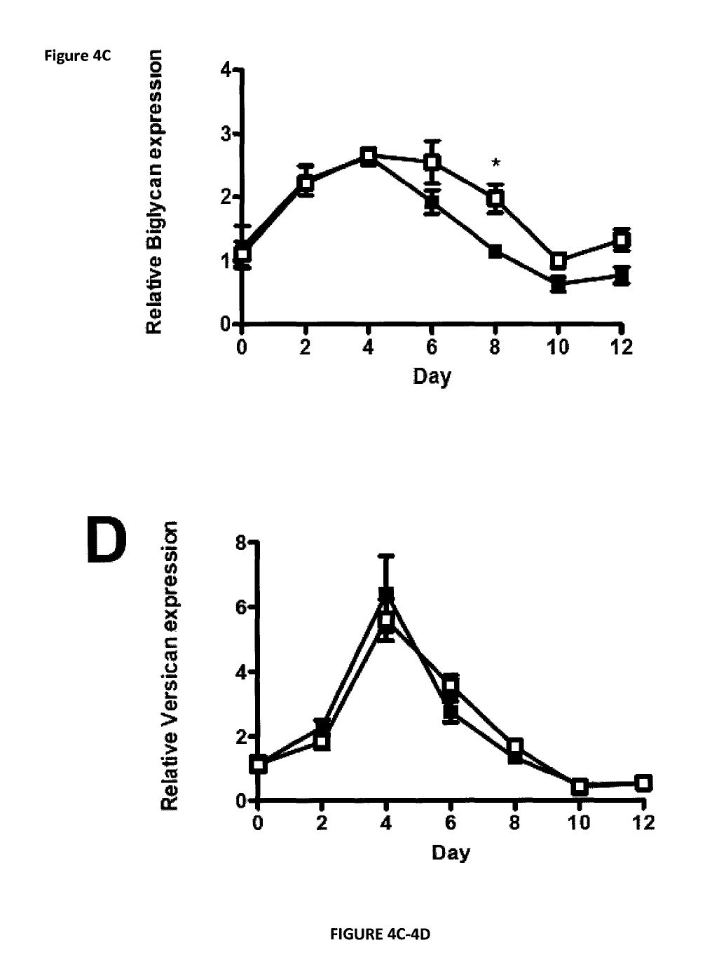

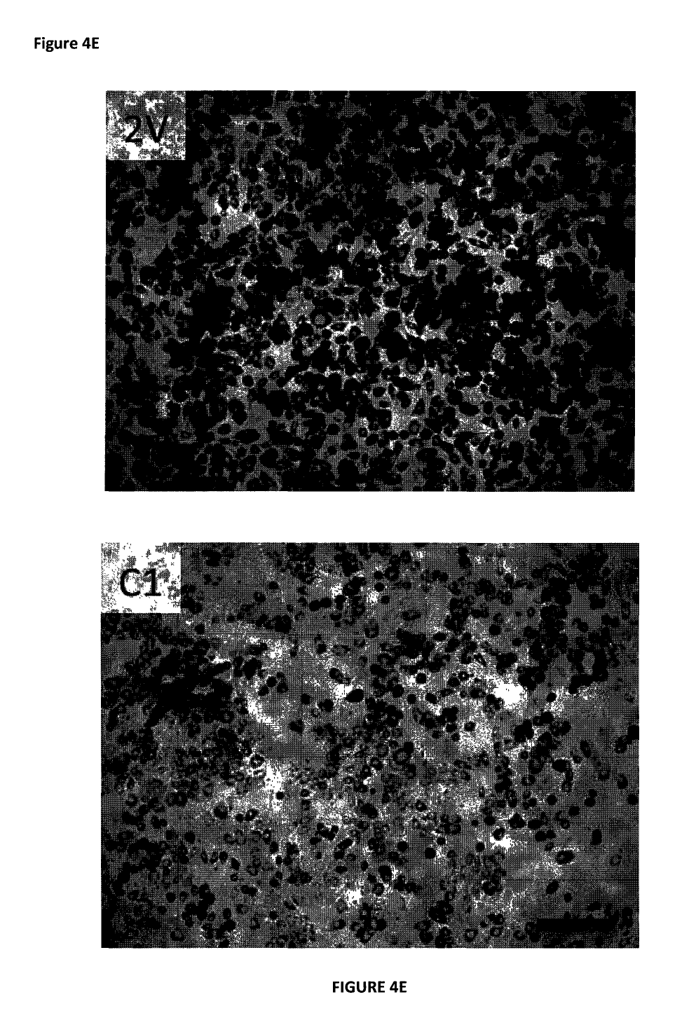

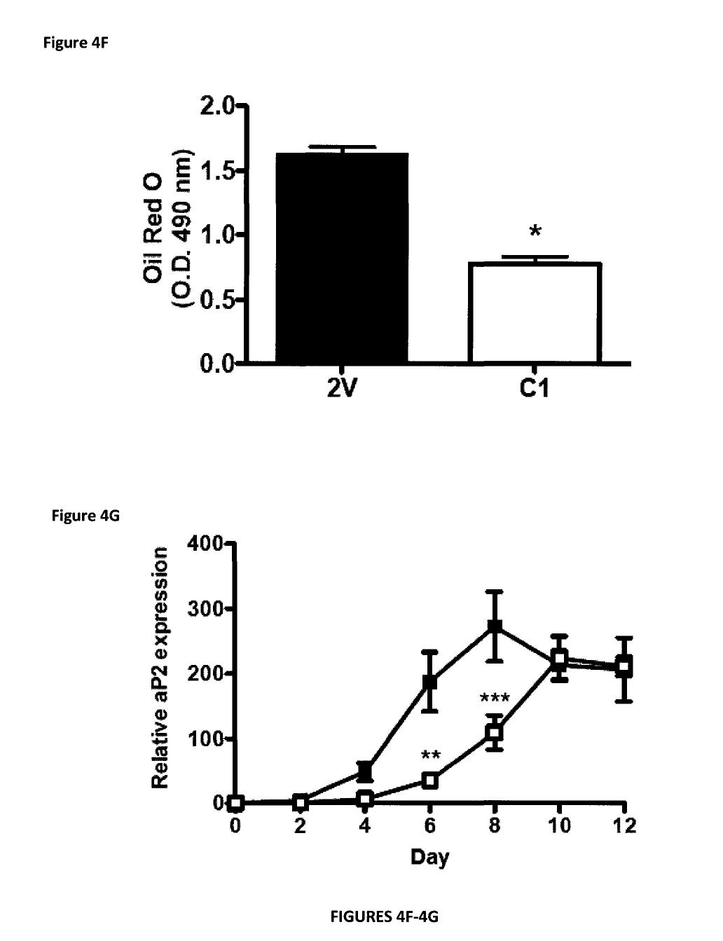

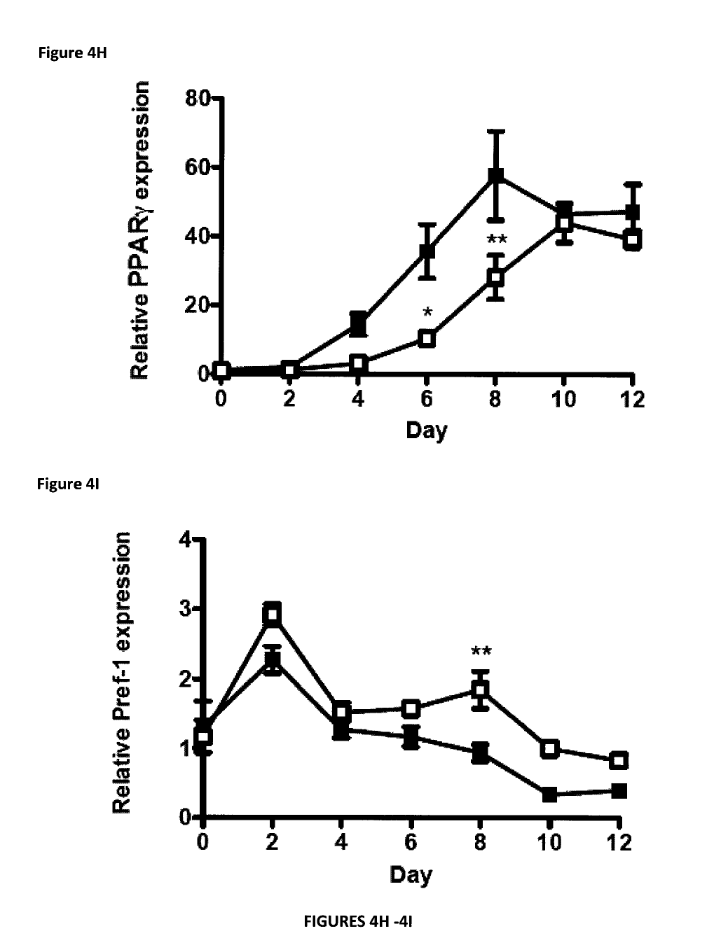

During differentiation of 3T3-F442A preadipocytes into mature adipocytes, mRNA expression of ADAMTS-5 increased (FIG. 4A) and that of ADAMTS-4 decreased (FIG. 4B) with time. ADAMTS-5 gene silencing using shRNA was confirmed by markedly reduced expression of ADAMTS-5 (FIG. 4A), without an effect on ADAMTS-4 expression (FIG. 4B). Biglycan (FIG. 4C) or versican (FIG. 4D) expression levels were not affected by ADAMST-5 gene silencing. Expression of aggrecan and brevican was not detected during the differentiation period.

ADAMTS-5 knock-down was associated with significantly impaired differentiation (FIG. 4E), as shown by reduced Oil Red O staining and quantitative analysis (FIG. 4F). This was further supported by lower expression levels of the adipogenic markers aP2 and PPARγ(FIG. 4G-H), and higher expression of the preadipocyte marker Pref-1 (FIG. 41). Trypan Blue staining did not reveal an effect of gene silencing on cell viability (cell death of 11.0±1.7% for clone Cl as compared to 9.8±0.1% for 2V control).

### Example 4

### Effect of ADAMTS5 on Differentiation of Embryonic Fibroblasts

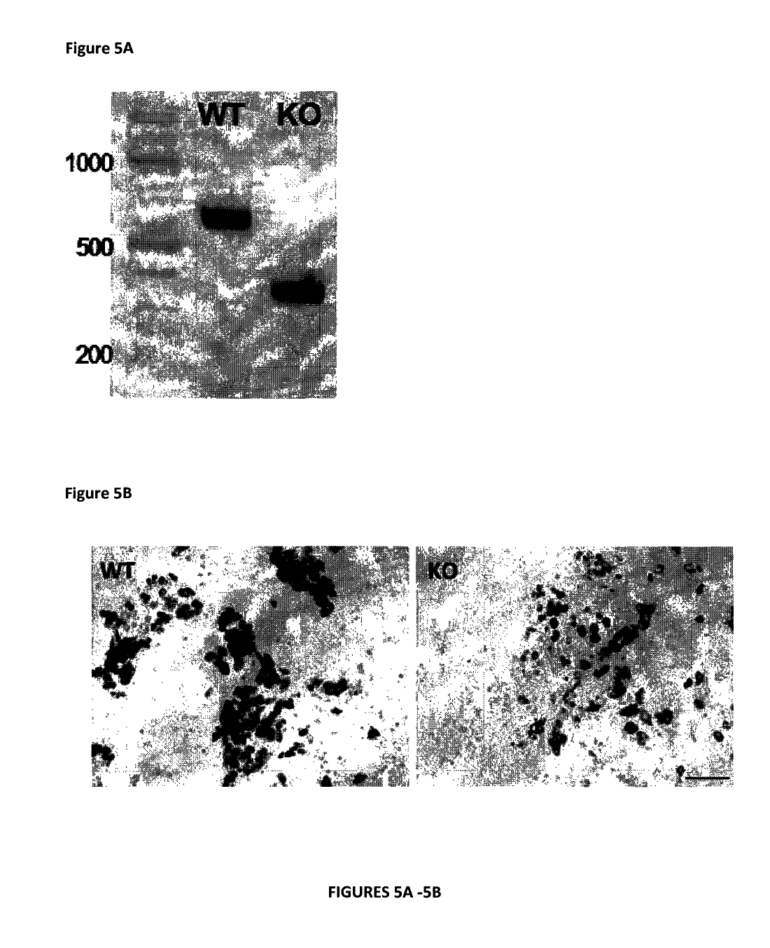

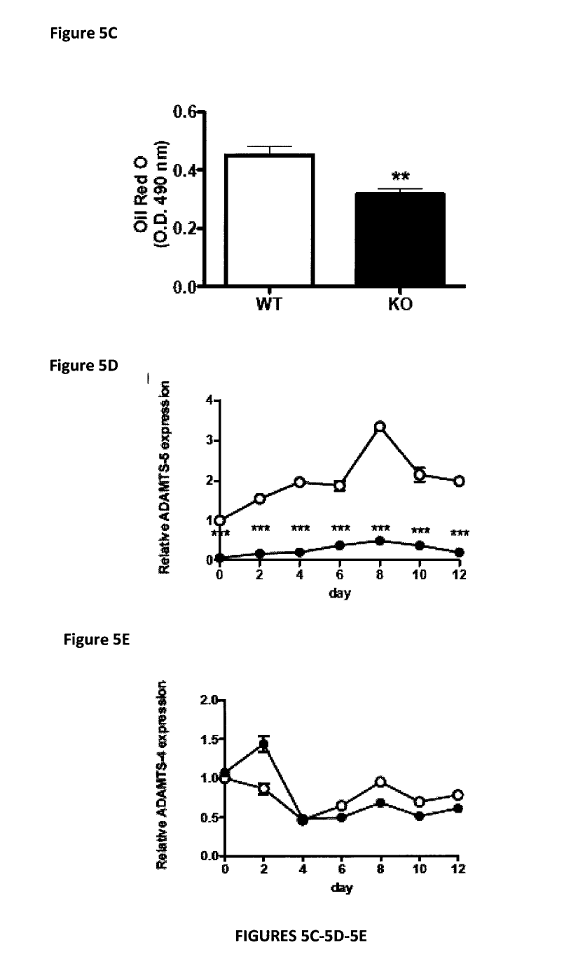

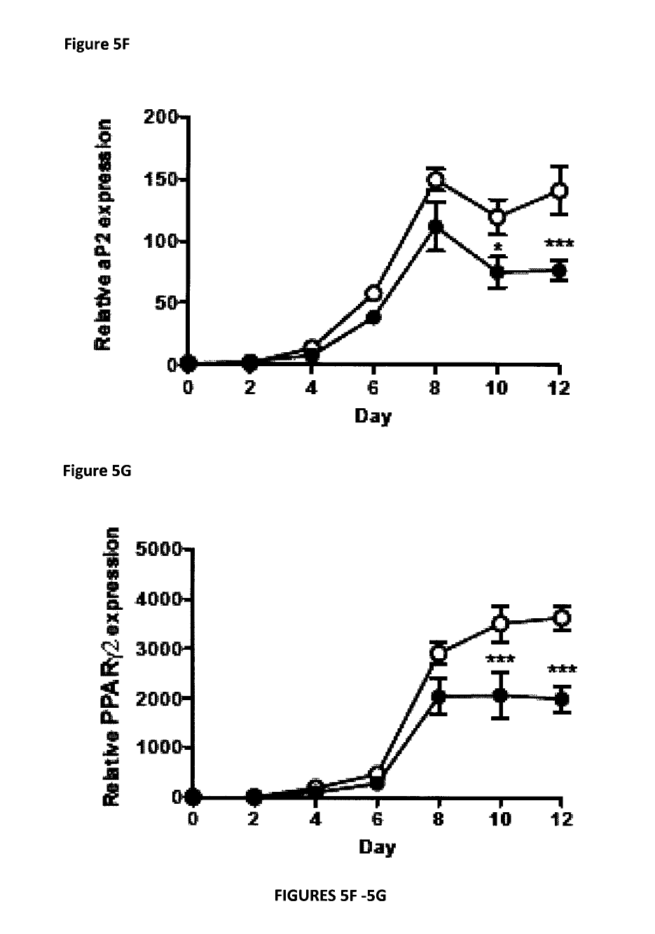

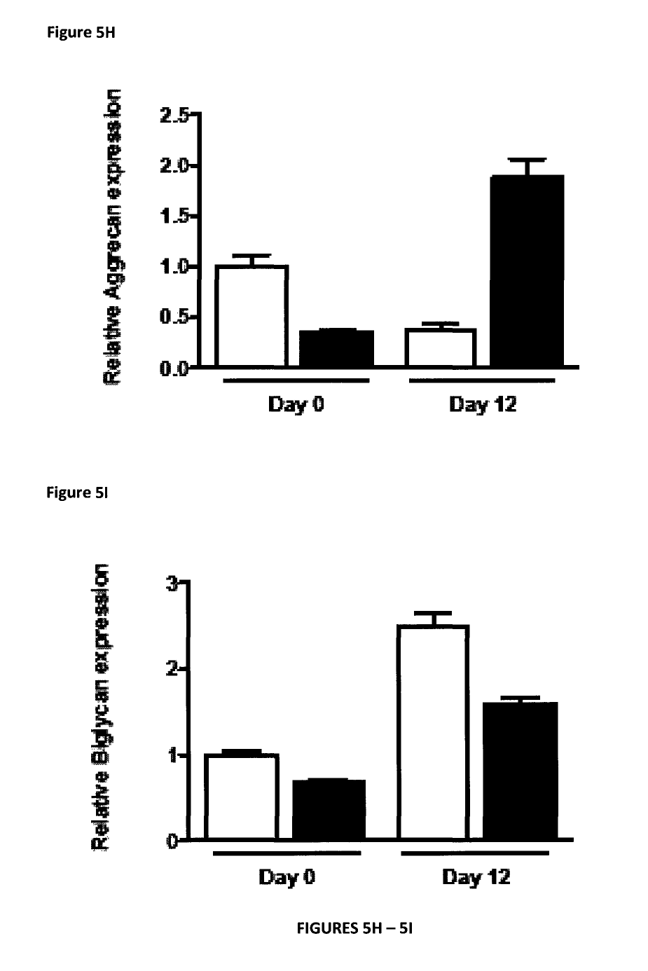

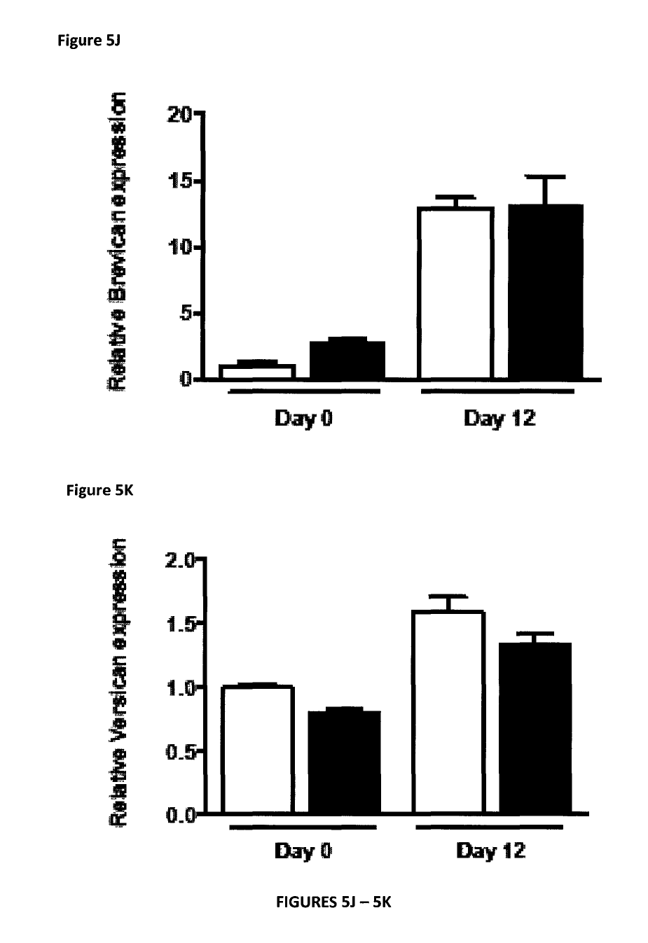

Genotyping of MEF derived from ADAMTS5^{+/+} and ADAMTS5^{-/-} cells confirmed the WT and homozygous deficient genotype (FIG. 5A). Differentiation of ADAMTS5^{-/-} MEF was markedly impaired as compared to MEF derived from ADAMTS5^{+/+} mice, as shown by Oil Red O staining after differentiation for 12 days (FIG. 5B). Quantification of the staining confirmed lower intracytoplasmatic lipid content in ADAMTS5^{-/-} MEF (FIG. 5C). ADAMTS5 expression during differentiation of ADAMTS5^{+/+} MEF peaked at day 8 (FIG. 5D). Expression of ADAMTS4 during differentiation of both ADAMTS5^{+/+} and ADAMTS5^{-/-} MEF peaked at day 2 for both genotypes, but was not different (FIG. 5E).

Impaired differentiation was further supported by significantly reduced expression of the adipogenic markers aP2 and PPARγ2 in ADAMTS5^{-/-} cells from day 10 on (FIG. 5F, G). Expression of aggrecan decreased during differentiation (day 12 versus day 0) for ADAMTS5^{+/+} MEF, but increased for ADAMTS5^{-/-} cells, resulting in higher expression levels in ADAMTS5^{-/-} as compared to ADAMTS5^{+/+} cells (FIG. 5H). Biglycan expression increased during differentiation of both genotypes, and was lower for ADAMTS5^{-/-} as compared to ADAMTS5^{+/+} MEF (FIG. 51). Expression of brevican (FIG. 5J) and versican (FIG. 5K) also increased during differentiation of both ADAMTS5^{+/+} and ADAMTS5^{-/-} MEF, but was not different between genotypes.

### Example 5

### De novo Adipogenesis in vivo

Following injection of 3T3-F442A preadipocytes with or without ADAMTS5 knock-down in the back of NUDE mice, de novo fat pads developed after 4 weeks of high fat diet feeding. Weight gain was comparable for all mice, resulting in comparable body weights for the knock-down and control cells (Table 4). The weights of SC and GN fat pads were comparable for both groups, whereas the de novo formed fat pads were significantly smaller when derived from the cells with ADAMTS5 knock-down.

Expression of Pref-I was 1.6-fold higher (p=0.03) in de novo fat pads derived from cells with ADAMTS5 knock-down, compatible with less differentiation and higher preadipocyte content.

### Example 6

### Effect of ADAMTS5 on WAT and BAT Development in a Murine Model of Nutritionally Induced Obesity

After 15 weeks of HFD, isolated subcutaneous (SC) WAT mass was not significantly different for both genotypes on either SFD or HFD, whereas gonadal (GN) WAT mass was lower for ADAMTS5^{-/-} mice kept on HFD, but not on SFD. (Table 1).

Surprisingly, ADAMTS5^{-/-} mice developed significantly more BAT than WT counterparts, as well on SFD as on HFD (Table 1).

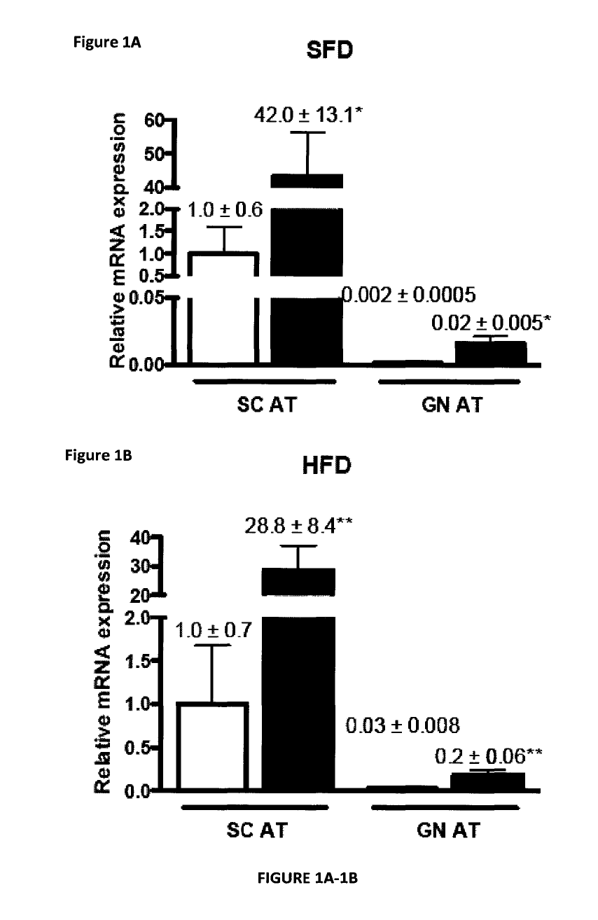

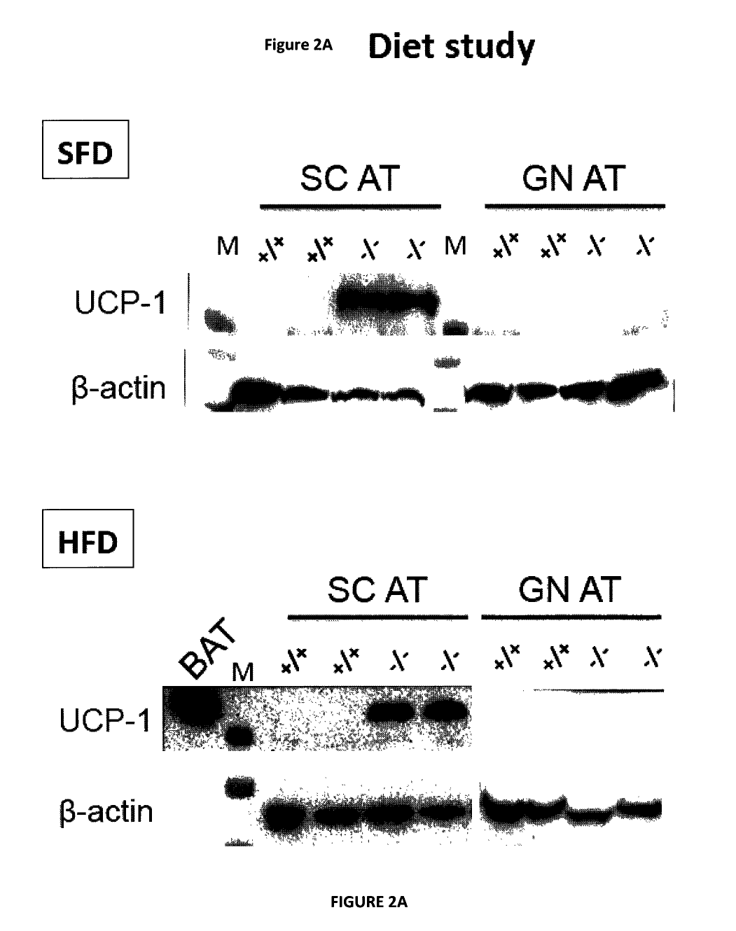

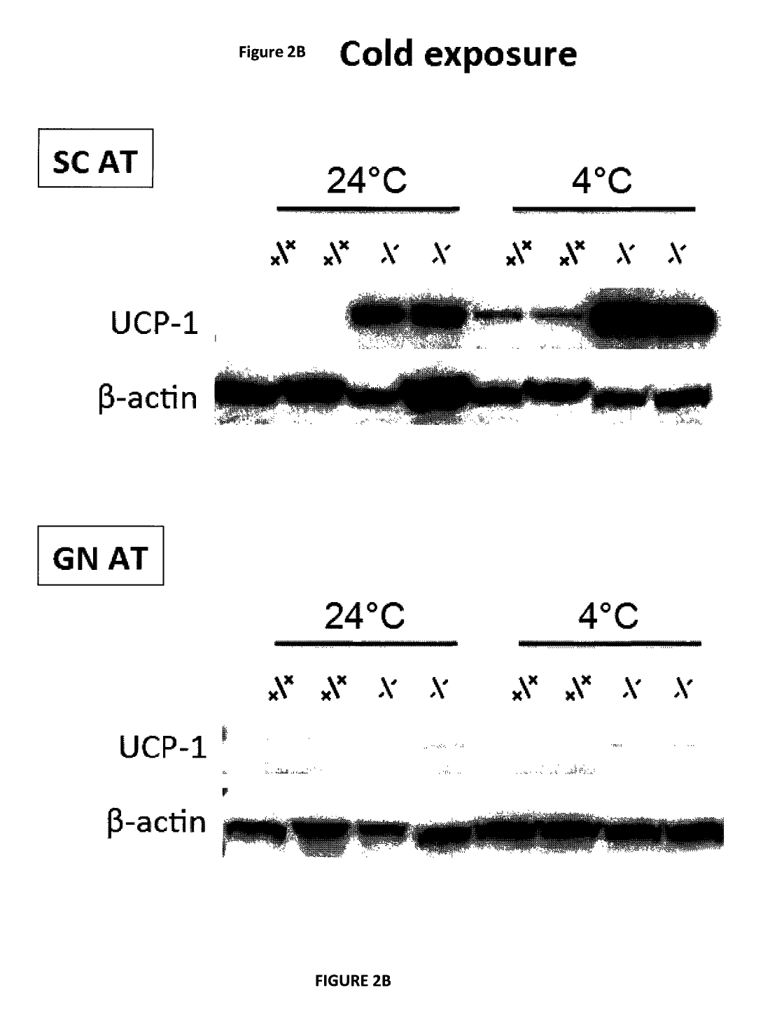

Analysis of gene expression levels of BAT markers (including UCP-1, PGC1α PRDM16, and Cidea) revealed that: 1) all markers are expressed in much higher level in BAT as compared to SC or GN WAT; and 2) both on SFD and HFD, all markers are expressed at significantly higher level in SC as well GN WAT of ADAMTS5^{-/-} as compared to WT mice (Table 2). This is illustrated in FIG. 1 for UCP-1. Furthermore, Western Blotting with protein extracts confirms abundance of UCP-1 in SC WAT of ADAMTS5^{-/-} versus WT mice (HFD) (FIG. 2A). For GN WAT, UCP-1 is much less present.

These findings confirm higher BAT mass and more browning (in particular in SC WAT) for ADAMTS5^{-/-} fat as compared to WT.

In a separate study, it was found that feeding with a more sugar-rich diet (MCC) for 4 weeks did result in reduced adiposity in ADAMTS5^{-/-} mice. Thus, for ADAMTS5^{-/-} mice (n=10) versus WT mice (n=9) total body weight gain was lower (4.7±0.3 vs 7.9±1.1 g; p=0.001), as well as weight of SC (452±27 vs 736±94 mg; p=0.02) and GN (1006±59 vs 1533±165; p=0.01) fat.

### Example 7

### Effect of ADAMTS5 on Browning of WAT Upon Cold Exposure

After 2 weeks of SFD and cold exposure, SC WAT mass of ADAMTS−/−mice was somewhat higher than that of WT mice, whereas GN WAT mass was not different, and BAT mass was significantly higher (Table 3).

Western blotting with protein extracts (FIG. 2B) showed that UCP-1 was detected in SC WAT of ADAMTS5−/− but not of WT mice kept at 24° C. After cold exposure, UCP-1 was also detected in the SC WAT of WT mice, but was much more abundant in that of ADAMTS5−/− mice.

In GN WAT, UCP-1 again was present at much lower level.

These findings confirm lower body weight in ADAMTS5−/− mice exposed to the cold, higher BAT mass and more pronounced browning of WAT, as compared to WT controls.

### Example 8

### Effect of ADAMTS5 on Liver Steatosis

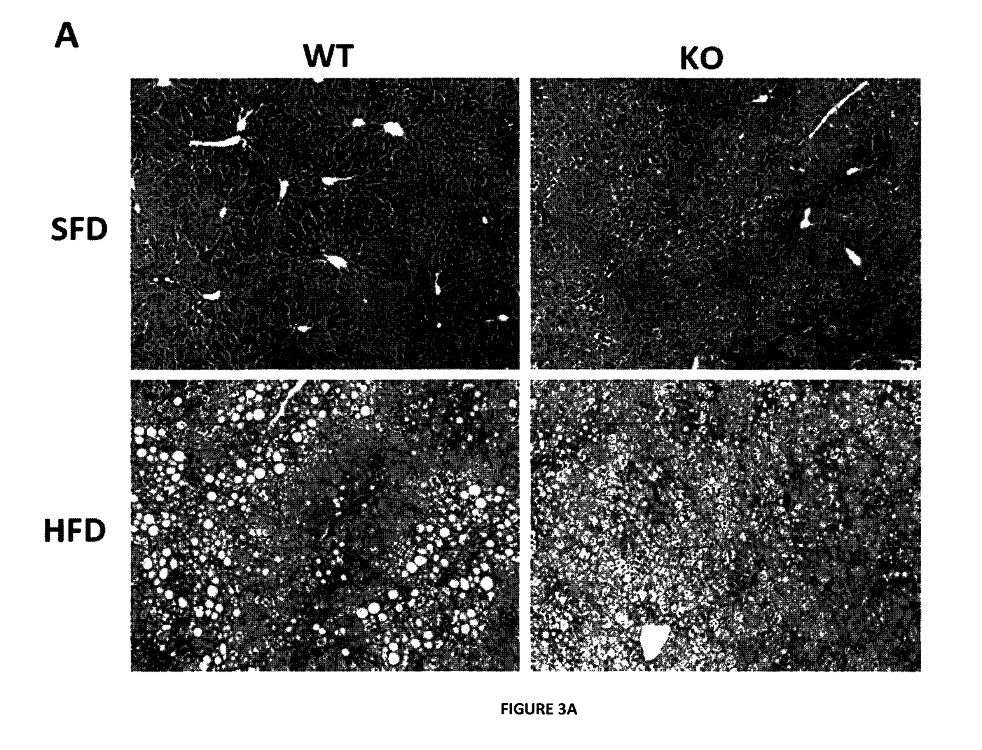

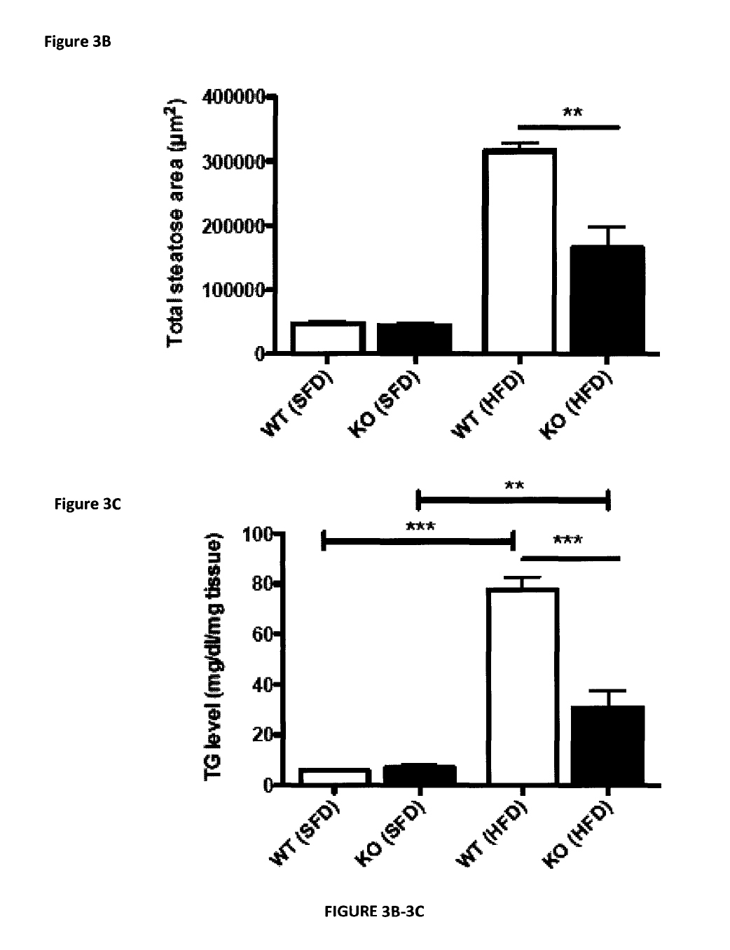

Haematoxylin/eosin staining of liver sections revealed less steatosis in the liver of ADAMTS5^{-/-} as compared to WT mice, after 15 weeks of HFD (FIG. 3A, B). Triglyceride levels in liver extracts (FIG. 3C) were significantly lower for ADAMTS5^{-/-} mice on HFD. This suggests that neutralization or depletion of ADAMTS5 may limit liver steatosis upon high fat diet feeding.

### DRAWING DESCRIPTION

### BRIEF DESCRIPTION OF THE DRAWINGS

FIG. 1: UCP-1 expression in SC and GN WAT of WT (white bars) and ADAMTS5−/− (black bars) mice kept on SFD (A) or HFD (B) for 15 weeks.

Data are means ±SEM of 8 to 12 determinations. Values are corrected for the housekeeping gene β-actin and subsequently normalized to SC WAT of WT mice on either SFD or HFD. *p<0.05 and **p<0.01 for ADAMTS5−/− versus WT.

FIG. 2: Western blotting for UCP-1 with protein extracts of SC or GN WAT of WT (+/+) and ADAMTS5 deficient (−/−) mice.

(A)Diet study: WT and ADAMTS−/− mice were kept on SFD or HFD for 15 weeks. Sample of BAT is applied at 200-fold lower concentration that those of SC and GN WAT.

(B)Cold exposure: WT and ADAMTS−/− mice on SFD were kept for 2 weeks at 24° C. or at 4° C.

β-actin was used as internal standard. Two representative samples are shown for each group.

FIG. 3: Effect of ADAMTS5 on liver steatosis. Liver sections of WT or ADAMTS5 deficient (KO) mice kept on SFD or HFD for 15 weeks are stained with haemotoxylin/eosin (A) and total steatose area (B) as well as liver triglyceride (TG) content (C) were quantified. *P<0.05

FIG. 4: Effect of ADAMTS5 gene silencing on in vitro differentiation of 3T3-F442A preadipocytes. (A-D) Time course of the expression of ADAMTS5 (A), ADAMTS4 (B), biglycan (C) and versican (D) during differentiation of 3T3-F442A cells with shRNA against ADAMTS5 (open squares) or scrambled shRNA (black squares). (E) Oil Red O staining at day 12. The scale bar corresponds to 200 μm. (F) Quantification of Oil Red O staining. (G-I) Time course of the expression of the adipogenic markers aP2 (G), PPARγ (H) and Pref←1 (I). Data are means ±SEM of 3 independent experiments. *p<0.05, **p<0.01, ***p<0.001 versus control.

FIG. 5: Differentiation of murine embryonic fibroblasts into mature adipocytes. (A) PCR genotyping of MEF derived from ADAMTS5+/+ (WT, 642 bp) or ADAMTS5−/− (KO, 374 bp) mice. Oil Red O staining (B) and quantification (C) of WT and KO MEF at day 12 of differentiation. The scale bar in panel (B) corresponds to 200 μm. (D-G) Time course of the expression of ADAMTS5 (D) or ADAMTS4 (E) and the adipogenic markers aP2 (F) and PPARγy2 (G) during differentiation ADAMTS5+/+ (open circles) and ADAMTS5−/− (closed circles) MEF. (H-K) Gene expression profile of aggrecan (H), biglycan (I), brevican (J) and versican (K) at days 0 and 12. Data are means ±SEM of 2 independent experiments performed in triplicate. *p<0.05, **p<0.01, *** p<0.001 versus control.

TABLE 1
Effect of ADAMTS-5 deficiency on organ weight and fat mass of mice kept on
standard fat diet (SFD) or high fat diet (HFD) for 15 weeks.

| | SFD | | HFD | |
|---|---|---|---|---|
| | ADAMTS-5^{+/+} | ADAMTS-5^{-/-} | ADAMTS-5^{+/+} | ADAMTS-5^{-/-} |
| | (n = 13) | (n = 12) | (n = 9) | (n = 11) |
| SC fat weight (mg) | 297 ± 21.0 | 306 ± 38.5 | 1428 ± 80.5 | 1198 ± 88.3 |
| GN fat weight (mg) | 655 ± 47.5 | 582 ± 89.9 | 2274 ± 93.5 | 1998 ± 72.9* |
| BAT (mg) | 87 ± 4.5 | 140 ± 13.1*** | 266 ± 28.3 | 428 ± 32.6** |
| Liver (mg) | 1191 ± 27.7 | 1399 ± 42.3** | 3852 ± 292 | 2203 ± 201*** |
| Pancreas (mg) | 341 ± 18.4 | 391 ± 14.5 | 280 ± 14.6 | 378 ± 13.7*** |
| Spleen (mg) | 75 ± 4.9 | 83 ± 5.2 | 117 ± 13.1 | 103 ± 4.9 |
| Lung (mg) | 168 ± 6.7 | 176 ± 8.2 | 213 ± 24.7 | 189 ± 10.9 |
| Heart (mg) | 172 ± 4.6 | 212 ± 8.9** | 179 ± 7.4 | 219 ± 9.0** |
| Kidneys (mg) | 398 ± 11.1 | 499 ± 26.4** | 447 ± 20.8 | 486 ± 12.8 |
| Brain (mg) | 452 ± 6.1 | 463 ± 8.8 | 456 ± 9.8 | 460 ± 6.0 |

Data are means ± SEM of 9-13 experiments in each group.
*p < 0.05,
**p < 0.01,
***p < 0.001 versus ADAMTS-5^{+/+} mice kept on the corresponding diet
SC, subcutaneous;
GN, gonadal;
BAT, brown adipose tissue

TABLE 2
Effect of ADAMTS5 deficiency on expression of “browning” markers in adipose
tissues of mice kept on standard fat diet (SFD) or high fat diet (HFD) for 15 weeks

| | ADAMTS5^{+/+} | | | ADAMTS5^{-/-} | | |
|---|---|---|---|---|---|---|
| | SC | GN | BAT | SC | GN | BAT |
| | SFD | | | | | |
| UCP-1 | 1.0 ± 0.6 | 0.002 ± 0.0005 | 1185 ± 80.2 | 42.0 ± 13* | 0.02 ± 0.005* | 1141 ± 94.4 |
| UCP-2 | 1.0 ± 0.09 | 1.1 ± 0.07 | 1.5 ± 0.06 | 2.5 ± 0.1*** | 3.2 ± 0.1*** | 0.5 ± 0.02*** |
| Cidea | 1.2 ± 0.25 | 0.04 ± 0.005 | 268 ± 9.6 | 15.5 ± 3.7*** | 0.2 ± 0.03*** | 212 ± 9.2** |
| Pgc1a | 1.05 ± 0.1 | 0.4 ± 0.06 | 12.3 ± 1.7 | 2.2 ± 0.29* | 0.93 ± 0.16* | 12.2 ± 2.2 |
| Prdm16 | 1.04 ± 0.1 | 0.5 ± 0.03 | 3.8 ± 0.4 | 1.50 ± 0.09* | 0.65 ± 0.05** | 2.8 ± 0.4 |
| | HFD | | | | | |
| UCP-1 | 1.0 ± 0.7 | 0.03 ± 0.008 | 2887 ± 120.6 | 28.8 ± 8.4** | 0.2 ± 0.06** | 3184 ± 450 |
| UCP-2 | 1.0 ± 0.07 | 2.5 ± 0.1 | 0.5 ± 0.02 | 3.3 ± 0.3*** | 4.2 ± 0.2*** | 2.5 ± 0.3*** |
| Cidea | 1.1 ± 0.2 | 0.03 ± 0.002 | 114 ± 5.5 | 4.9 ± 0.6*** | 0.2 ± 0.02*** | 126 ± 18 |
| Pgc1a | 1.06 ± 0.15 | 0.21 ± 0.03 | 8.2 ± 0.62 | 2.1 ± 0.25** | 0.6 ± 0.1** | 11.6 ± 1.6 |
| Prdm16 | 1.02 ± 0.07 | 0.28 ± 0.05 | 1.61 ± 0.077 | 1.3 ± 0.091* | 0.6 ± 0.05*** | 2.4 ± 0.3* |

Data are means ± SEM of 6-12 determinations. Values are corrected for the housekeeping gene β-actin and subsequently normalized to SC AT of ADAMTS-5^{+/+} mice on SFD or HFD.
*p < 0.05,
**p < 0.01,
***p < 0.001 versus ADAMTS-5^{+/+} mice kept on the corresponding diet.
SC, subcutaneous;
GN, gonadal;
BAT, brown adipose tissue

TABLE 3
Effect of cold exposure on adipose tissues in wild-type (ADAMTS-5^{+/+}) and
ADAMTS-5 deficient (ADAMTS-5^{-/-}) mice.

| | 24° C. | | 4° C. (n = 6) | |
|---|---|---|---|---|
| | ADAMTS-5^{+/+} | ADAMTS-5^{-/-} | ADAMTS-5^{+/+} | ADAMTS-5^{-/-} |
| | (n = 3) | (n = 3) | (n = 6) | (n = 6) |
| Body weight (g) | | | | |
| Start | 20.8 ± 0.3 | 21.2 ± 0.4 | 21.0 ± 0.4 | 21.2 ± 0.5 |
| Week 1 | 22.1 ± 0.8 | 22.4 ± 0.5 | 22.2 ± 0.6 | 20.5 ± 0.6 |
| Week 2 | 22.7 ± 0.7 | 23.1 ± 0.6 | 22.6 ± 0.6 | 20.1 ± 0.8* |
| Food intake (g/day) | | | | |
| Week 1 | 3.9 ± 0.1 | 4.5 ± 0.01 | 8.4 ± 0.2 | 8.8 ± 0.4 |
| Week 2 | 3.7 ± 0.2 | 3.9 ± 0.1 | 9.0 ± 0.2 | 8.9 ± 0.4 |
| SC fat (mg) | 229 ± 13.2 | 226 ± 27.2 | 175 ± 13.6 | 235 ± 10.1* |
| GN fat (mg) | 396 ± 64.5 | 403 ± 95.5 | 220 ± 14.5 | 185 ± 19.5 |
| BAT (mg) | 64 ± 0.7 | 110 ± 6.7 | 82 ± 2.2 | 125 ± 13.2** |

Data are means ± SEM;
*p < 0.05,
**p < 0.01 versus ADAMTS-5^{+/+} mice kept on the corresponding temperature.
SC, subcutaneous;
GN, gonadal;
BAT, brown adipose tissue

TABLE 4
Effect of ADAMTS5 knock-down (KD) in 3T3-F442A preadipocytes
on de novo adipogenesis following injection in NUDE mice kept on
high fat diet for 4 weeks.

| | Control | ADAMTS5 KD |
|---|---|---|
| | (n = 4) | (n = 4) |
| Body weight start (g) | 18.1± 0.8 | 20.9 ± 0.8 |
| Body weight end (g) | 24.3 ± 0.6 | 25.1 ± 0.7 |
| SC fat weight (mg) | 206 ± 13.4 | 198 ± 18.0 |
| GN fat weight (mg) | 315 ± 17.7 | 348 ± 36.4 |
| de novo fat pad weight (mg) | 24.5 ± 1.3 | 14.1 ± 1.1* |

Data are means ± SEM;
*p < 0.01

1. A method for treating of an ADAMTS5 associated disorder in a subject by administering an ADAMTS5 inhibitor, wherein the ADAMTS5 associated disorder is an overweight disorder or a metabolic disorder which is obesity.

2. The method according to claim 1, wherein the ADAMTS5 associated disorder is an overweight disorder or a metabolic disorder selected from obesity, type II diabetes, insulin resistance, disorder associated with insufficient insulin activity, disrupted thermogenesis in overweight, liver triglyceride accumulation, hepatosteatosis, fatty liver and non-alcoholic fatty liver disease.

3. The method according to claim 1, whereby the ADAMTS5 inhibitor is an antigen binding protein comprising at least one immunoglobulin variable domain capable of binding to human ADAMTS5.

4. The method according to claim 1, whereby the inhibitor is selected from a specific polyclonal antibody, a monoclonal antibody, a full-length antibody, a binding fragment of an antibody and a surrogate of an antibody capable of binding to human ADAMTS5.

5. The method according to claim 1, whereby the inhibitor is a binding fragment of an antibody or a surrogate of an antibody selected from the group consisting of aFab′, F(ab′)2, Fab, Fv, vIgG, scFv fragments and surrobodies, rIgG, disulfide-stabilized Fv antibodies (dsFv), diabodies, triabodies, and single domain antibodies, such as a camelized antibody or nanobody or humanized camel or shark antibody or nanobody and capable of binding to human ADAMTS5.

6. The method according to claim 1, whereby the ADAMTS5 inhibitor is an ADAMTS5 antigen-binding fragment of a monoclonal antibody selected from a scFV, Fab, Fab2, F(ab′)2, Fv or dAB and capable of binding to human ADAMTS5.

7. The method according to claim 1, whereby the ADAMTS5 inhibitor is a chimeric or humanized antibody or antigen-binding fragment thereof and capable of binding to human ADAMTS5.

8. The method according to claim 1, whereby the ADAMTS5 inhibitor specifically binds to ADAMTS5.

9. The method according to claim 1, whereby the ADAMTS5 inhibitor specifically binds to human ADAMTS5.

10. The method according to claim 1, whereby inhibitor is a monoclonal antibody comprising a heavy chain comprising CDRH1, CDRH2 and CDRH3 and a light chain comprising CDRL1, CDRL2 and CDRL3, wherein the complementarity determining regions (CDRs) of the heavy chain are selected from the group of: a) CDRH1 having at least about 80% sequence identity to amino acid sequence DAWMD; b) CDRH2 having at least about 70% sequence identity to amino acid sequence EIRHKANDHAIFYXESVKG; and c) CDRH3 having at least about 70% sequence identity to amino acid sequence TYYYGSSYGYCDV or PFAY; and the complementarity determining regions of the light chain are selected from the group of: d) CDRL1 having at least about 70% sequence identity to amino acid sequence KASQSVGTTIV or RTSENIYSYLA; e) CDRL2 having at least about 70% sequence identity to amino acid sequence NAKTLAE or SASNRXT; and f) CDRL3 having at least about 70% sequence identity to amino acid sequence QQYSSYPFT or QHHYGTPWT.

11. The method according to claim 1, whereby the inhibitor is a monoclonal antibody comprising a heavy chain comprising CDRH1, CDRH2 and CDRH3 and a light chain comprising CDR1, CDRL2 and CDRL3, wherein the complementarity determining regions (CDRs) of the heavy chain are selected from: (a) CDRH1 is amino acid sequence DAWMD; (b) CDRH2 is select from amino acid sequence EIRHKANDHAIFYAESVKG, EIRNKANNHARHYAESVKG, EIRHKANDYAIFYDESVKG, EIRHKANDHAIFYDESVKG, or DIRNTANNHATFYAESVKG, EIRHKANDHAIFYDESVKG; and (c) CDRH3 is TYYYGSSYGYCDV or PFAY; and the complementarity determining regions of the light chain are selected from: (d) CDRL1 is select from amino acid sequence KASQSVGTTIV, RTSENIYSYLA, or KASQNVGTAVV; (e) CDRL2 is select from amino acid sequence NAKTLAE, SASNRHT, SASTRYT, or SASNRYT; and (f) CDRL3 is select from amino acid sequence QQYSSYPFT, QHHYGTPWT, QQYVNYPFT, or QQYTSYPFT.

12. The method according to claim 1, whereby the inhibitor is a monoclonal antibody comprising six CDRs wherein:
(SEQ ID NO: 2)
a) CDRH1 is DAWMD,
(SEQ ID NO: 13)
CDRH2 is EIRNKANNHARHYAESVKG,
and
(SEQ ID NO: 18)
CDRH3 is TYYYGSSYGYCDV
and
(SEQ ID NO: 20)
CDRL1 is RTSENIYSYLA,
(SEQ ID NO: 22)
CDRL2 is NAKTLAE
and
(SEQ ID NO: 27)
CDRL3 is QHHYGTPWT;
or
(SEQ ID NO: 2)
b) CDRH1 is DAWMD,
(SEQ ID NO: 15)
CDRH2 is EIRHKANDHAIFYDESVKG,
and
(SEQ ID NO: 5)
CDRH3 is PFAY
and
(SEQ ID NO: 19)
CDRL1 is KASQSVGTTIV,
(SEQ ID NO: 23)
CDRL2 is SASNRHT
and
(SEQ ID NO: 29)
CDRL3 is QQYTSYPFT.

13. (canceled).

14. The method according to claim 1, whereby the inhibitor is a nucleic acid molecule in a vector to express a therapeutic agent.

15. The method according to claim 1, whereby the ADAMTS5 inhibitor comprises an antibody, or antigen-binding fragment thereof, that:
a) binds to ADAMTS5 with a dissociation constant (K D) of 150 pM or less, as determined by real-time biospecific interaction analysis (BIA) using surface plasmon resonance (SPR) technology, or with an IC50 of 100 pM or less; or
b) binds to a neutralizing epitope of human ADAMTS5 with an affinity of at least about 5×10⁴ liter/mole as measured by an association constant (Ka).

16. (canceled).

17. The method according to claim 1, whereby the treatment of the subject induces one or more primary efficacy endpoints or direct efficacy endpoints of a) increase in thermogenesis in brown adipose tissue, b) increase in insulin sensitivity of skeletal muscle, white adipose tissue, or liver; c) increase in glucose tolerance, d) increase in basal respiration, maximal respiration rate, or uncoupled respiration, e) increase in metabolic rate, f) decrease in hepatosteatosis or g) decrease in baseline in body weight.

18. The method according to claim 1, which can modulate one or more of the following biological activities: thermogenesis in adipose cells, differentiation of adipose cells, and insulin sensitivity of adipose cells.

19. The method according to claim 1, wherein the treatment prevents or reduces the de novo adipogenesis or prevents or reduces de novo fat development.

20. The method according to claim 1, wherein the treatment impairs differentiation of adipocyte precursor cells into mature adipocytes (hyperplasia) in a mammalian subject.

21. The method according to claim 1, whereby the ADAMTS5 inhibitor is selected from the group consisting of a peptide, a peptidomimetic, an antibody, a double-stranded RNA, an aptamer, a small interfering RNA (siRNA), a peptide fragment of ADAMTS5 and a ribozyme

22. The method according to claim 21, whereby the peptide, peptidomimetic, antibody, double-stranded RNA, aptamer, small interfering RNA (siRNA), peptide fragment of ADAMTS5 or ribozyme therapy against ADAMTS5 is delivered by gene therapy and in vivo expressed in a subject.

23. (canceled).

24. An ADAMTS5 inhibitor selected from:
a. an antigen binding protein comprising at least one first immunoglobulin variable domain capable of binding to human ADAMTS5;
b. a nucleic acid molecule in a vector to express a therapeutic agent; and
c. a peptide, a peptidomimetic, an antibody, a double-stranded RNA, an aptamer, a small interfering RNA (siRNA), a peptide fragment of ADAMTS5 or a ribozyme.
